(19) United States
(12) Patent Application Publication
(10) Pub. No.: US 2024/0325209 A1
(43) Pub. Date: Oct. 3, 2024

(54) REDUCED PRESSURE APPARATUSES AND METHODS

(71) Applicant: T.J.Smith and Nephew,Limited, Hull (GB)

(72) Inventors: Adam John Fisher, Hull (GB); Benjamin James Gardner, Hull (GB); Daniel Jorna, Hull (GB); Camilo Patrick Madriz, Bristol (GB); Daniel Lee Steward, Hull (GB); Liam Bradley James Thomson, Hull (GB); Rhianna Ward, Brough (GB); Fraser George Weedon, Hull (GB)

(21) Appl. No.: 18/577,528

(22) PCT Filed: Jul. 14, 2022
(86) PCT No.: PCT/EP2022/069767

§ 371 Date: Jan. 8, 2024

(30) Foreign Application Priority Data
Jul. 16, 2021 (GB) 2110240.5

(51) Int. Cl. A61F 13/05; A61F 13/15; A61M 1/00

(52) U.S. Cl. CPC A61F 13/05; A61M 1/962; A61M 1/964; A61F 2013/15008; A61M 2205/587; A61M 2205/75

(57) ABSTRACT

Disclosed herein are embodiments of a wound treatment apparatus with electronic components integrated within a wound dressing. In some embodiments. a wound dressing apparatus can comprise a wound dressing. The wound dressing can comprise an absorbent material. an electronics unit comprising a negative pressure source. the electronics unit integrated within the wound dressing and at least partially encapsulated by one or more layers of a flexible film. At least one of the layers of the flexible film can comprise a window or aperture configured to permit fluid communication between the absorbent material and the negative pressure source.

### CROSS-REFERENCE TO RELATED APPLICATIONS

This application claims priority to U.K. Patent Application No. 2110240.5, filed on Jul. 16, 2021, entitled “REDUCED PRESSURE APPARATUSES AND METHODS,” which is hereby incorporated by reference in its entirety and made part of this disclosure.

### TECHNICAL FIELD

Embodiments described herein relate to apparatuses, systems, and methods the treatment of wounds, for example using dressings in combination with negative pressure wound therapy.

### DESCRIPTION OF THE RELATED ART

The treatment of open or chronic wounds that are too large to spontaneously close or otherwise fail to heal by means of applying negative pressure to the site of the wound is well known in the art. Negative pressure wound therapy (“NPWT”) systems currently known in the art commonly involve placing a cover that is impermeable or semi-permeable to fluids over the wound, using various means to seal the cover to the tissue of the patient surrounding the wound, and connecting a source of negative pressure (such as a vacuum pump) to the cover in a manner so that negative pressure is created and maintained under the cover. It is believed that such negative pressures promote wound healing by facilitating the formation of granulation tissue at the wound site and assisting the body's normal inflammatory process while simultaneously removing excess fluid, which may contain adverse cytokines and/or bacteria. However, further improvements in NPWT are needed to fully realize the benefits of treatment.

### SUMMARY

Embodiments of the present disclosure relate to apparatuses and methods for wound treatment. Some of the wound treatment apparatuses described herein comprise a negative pressure source or a pump system for providing negative pressure to a wound. Wound treatment apparatuses may also comprise wound dressings that may be used in combination with the negative pressure sources and pump assemblies described herein. In some embodiments, a negative pressure source is incorporated into a wound dressing apparatus so that the wound dressing and the negative pressure source are part of an integral or integrated wound dressing structure that applies the wound dressing and the negative pressure source simultaneously to a patient's wound. The negative pressure source and/or electronic components may be positioned between a wound contact layer and a cover layer of the wound dressing. An electronics assembly can be incorporated into a protective enclosure formed at least in part by a flexible film or a plurality of flexible films. These and other embodiments as described herein are directed to overcoming particular challenges involved with incorporating a negative pressure source and/or electronic components into a wound dressing.

In one aspect, a wound dressing apparatus can comprise a cover layer configured to cover and form a seal over a wound; and an electronics assembly comprising: an electronics unit comprising a negative pressure source, wherein the negative pressure source comprises a body portion and an inlet extending from the body portion, wherein the inlet comprises a first end opposite a second end, wherein the second end is attached to the body portion; and a housing comprising a plurality of flexible film layers, wherein the electronics unit is at least partially enclosed within the plurality of flexible film layers, wherein the plurality of flexible film layers comprises a first flexible film layer and a second flexible film layer, wherein the first and second flexible film layers are bonded together along a periphery of each of the first and second flexible film layers to at least partially enclose the electronics unit between the first and second flexible film layers, wherein the first flexible film layer comprises an aperture configured to receive the inlet and form a hermetic seal around the inlet to prevent wound exudate from entering into the electronics unit; wherein the cover layer comprises an opening configured to receive the electronics assembly.

The wound dressing apparatus of any of the preceding paragraphs and/or any of the apparatuses, systems, or devices disclosed herein can include one or more of the following features. The first end of the inlet can comprise a first diameter and the second end of the inlet comprises a second diameter. The second diameter of the second end can be greater than the first diameter of the first end so that the inlet tapers from the second end to the first end. The wound dressing apparatus can further comprise: a wound contact layer comprising a proximal wound-facing face and a distal face, wherein the proximal wound-facing face is configured to be positioned in contact with the wound; at least one absorbent layer over the wound contact layer; wherein the cover layer is configured to cover and form a seal over the wound contact layer and the at least one absorbent layer; and wherein the at least one absorbent layer comprises a recess configured to receive the electronics assembly. The electronics unit can further comprise: an outlet or exhaust mechanism positioned on an outlet of the negative pressure source, the outlet or exhaust mechanism comprising a vent aperture configured to expel air exhausted from the negative pressure source; and a flexible circuit board, wherein the flexible circuit board comprises one or more of a sensor, a switch, a vent hole, and/or a light or LED indicators. The first flexible film layer can comprise a second aperture configured to be aligned with the sensor on the circuit board, wherein the sensor is configured to measure the pressure from the inlet of the negative pressure source. The wound dressing apparatus can further comprise a filter configured to be positioned between the second aperture and the first sensor. The first flexible film layer and the second flexible film layer can comprise a waterproof and/or gas tight film material. The first flexible film layer and the second flexible film layer can comprise a polyurethane, a thermoplastic polyurethane, polyester, ethylene-vinyl acetate, or polyethylene. The first flexible film layer and the second flexible film layer can comprise a material with a moisture vapor permeability configured to allow vapor to pass through the first and second flexible film layers. The housing can further comprise an upper housing portion comprising a material layer forming a label for the electronics assembly, the upper housing portion comprises a first wound facing side and an opposite second side, wherein the second flexible film layer is bonded to the first side of the upper housing portion.

In one aspect, the wound dressing apparatus can comprise: a cover layer configured to cover and form a seal over a wound; and an electronics assembly comprising: an electronics unit comprising a negative pressure source; and a housing comprising: a lower flexible film layer comprising a first wound facing surface and an opposite second surface; an upper housing portion comprising a first wound facing surface and an opposite second surface, the upper housing portion comprising a coating along a perimeter of the first surface of the upper housing portion; wherein the electronics unit is at least partially enclosed within the flexible film layer and the upper housing portion; and wherein the flexible film layer is bonded to the coating of the upper housing portion along a perimeter of the flexible film layer to at least partially enclose the electronics unit between the flexible film layer and the upper housing portion; wherein the cover layer comprises an opening configured to receive the electronics assembly.

The wound dressing apparatus of any of the preceding paragraphs and/or any of the apparatuses, systems, or devices disclosed herein can include one or more of the following features. The flexible film layer can be bonded to the coating of the upper housing portion through heat and/or pressure. The wound dressing apparatus can further comprise a circuit board comprising an exhaust hole and a first wound facing surface and an opposite second surface. The coating of the upper housing portion can comprise a first coating, wherein the circuit board comprises a second coating along a perimeter of the second surface of the circuit board, and wherein the first coating is bonded or sealed to the perimeter of the flexible film layer and the second coating on the circuit board. The coating of the upper housing portion can comprise a first coating, the upper housing portion comprises a second coating on the first surface of the upper housing portion, wherein the second coating surrounds an exhaust hole in the upper housing portion, wherein the circuit board comprises a third coating on the second surface of the circuit board, the third coating surrounds the exhaust hole in the circuit board, and wherein the second coating of the upper housing portion is bonded to the third coating on the circuit board. The coating of the upper housing portion can comprise a first coating, the upper housing portion comprises a second coating on the first surface of the upper housing portion, wherein the second coating surrounds an exhaust hole in the upper housing portion, wherein the circuit board comprises a third coating on the second surface of the circuit board, the third coating surrounds the exhaust hole in the circuit board, wherein the circuit board comprises a fourth coating along a perimeter of the second surface of the circuit board, and wherein the second coating of the upper housing portion is bonded to the third coating on the circuit board and the first coating is bonded to the perimeter of the flexible film layer and the fourth coating on the circuit board. The negative pressure source can comprise a body portion and an inlet extending from the body portion, wherein the inlet comprises a first end opposite a second end, wherein the second end is attached to the body portion. The flexible film layer can comprise an aperture configured to receive the inlet and form a hermetic seal around the inlet to prevent wound exudate from entering into the electronics unit. The coating can comprise a dispersion coating. The coating can comprise a polyurethane (PU) dispersion coating.

Any of the features, components, or details of any of the arrangements or embodiments disclosed in this application, including without limitation any of the pump embodiments and any of the negative pressure wound therapy embodiments disclosed below, are interchangeably combinable with any other features, components, or details of any of the arrangements or embodiments disclosed herein to form new arrangements and embodiments.

### BRIEF DESCRIPTION OF THE DRAWINGS

FIGS. 1A-1C illustrate a wound dressing incorporating a source of negative pressure and/or other electronic components within the wound dressing;

FIGS. 2A-2B illustrate an electronics unit that may be incorporated into a wound dressing;

FIG. 3 is an exploded perspective view of an electronics assembly enclosing an electronics unit within a housing;

FIG. 4A illustrates a bottom perspective view of the electronics assembly of FIG. 3;

FIG. 4B illustrates a top perspective view of the electronics assembly of FIG. 3;

FIG. 5A is an exploded view of a wound dressing incorporating an electronics assembly within the wound dressing layers;

FIG. 5B illustrates a cross sectional layout of the material layers of a wound dressing incorporating an electronics assembly within the dressing;

FIG. 6 is an exploded perspective view of another configuration of an electronics assembly enclosing an electronics unit within a housing;

FIG. 7A is an exploded perspective view of a flexible film layer and a filter of the electronics assembly shown in FIG. 6;

FIG. 7B is a perspective view of the flexible film layer and the filter of the electronics assembly shown in FIG. 7A;

FIG. 8A is a top view of another configuration of an inlet protection mechanism;

FIG. 8B is a perspective view of a component of the inlet protection mechanism shown in FIG. 8A;

FIG. 9A is an exploded view of a top portion of the electronics assembly shown in FIG. 6;

FIGS. 9B-9C are bottom perspective views of the top portion shown in FIG. 9A;

FIG. 9D is top perspective views of the top portion shown in FIG. 9A;

FIG. 9E is a top perspective view of the electronics assembly shown in FIG. 6 with the label partially removed;

FIG. 10 is a top perspective view of another configuration of a label;

FIG. 11 is a top perspective view of the electronics assembly shown in FIG. 6 without the label;

FIG. 12A illustrates a top view of the label of the electronics assembly shown in FIG. 6;

FIGS. 12B and 12D illustrate cross-sectional views of the label shown in FIG. 12A;

FIGS. 12C and 12E illustrate close-up views of embossed portions of the label shown in FIG. 12A;

FIG. 13A illustrates a top view of another configuration of a label;

FIGS. 13B and 13D illustrate cross-sectional views of the label shown in FIG. 13A;

FIGS. 13C and 13E illustrate close-up views of embossed portions of the label shown in FIG. 13A;

FIG. 14A is a bottom view of the inlet protection mechanism, the negative pressures source, and the exhaust mechanism of the electronics assembly shown in FIG. 6;

FIG. 14B is a top perspective view of the inlet protection mechanism, the negative pressures source, and the exhaust mechanism shown in FIG. 14A;

FIG. 15A is a bottom view of the exhaust mechanism of the electronics assembly shown in FIG. 6;

FIG. 15B is a top perspective view of the exhaust mechanism shown in FIG. 15A;

FIGS. 16A-16H illustrate a portion of a process for manufacturing the electronics assembly shown in FIG. 6 using a manufacturing tool;

FIG. 17 is bottom perspective view of the electronics assembly shown in FIG. 6 within another manufacturing tool.

FIG. 18 illustrates an exploded perspective view of another configuration of an electronics assembly enclosing an electronics unit within a housing.

FIG. 19 illustrates an example of a label that can be used with the electronic assembly.

FIG. 20A-20B illustrates a configuration of a lower side of a label (FIG. 20A) and an upper side of a circuit board (FIG. 20B).

FIG. 21A-21B illustrates a configuration of the lower side of a label (FIG. 21A) and an upper side of a circuit board (FIG. 21B).

FIG. 22A-22B illustrates a configuration of the lower side of a label (FIG. 22A) and the upper side of a circuit board (FIG. 22B).

### DETAILED DESCRIPTION

Embodiments disclosed herein relate to apparatuses and methods of treating a wound with reduced pressure, including a source of negative pressure and wound dressing components and apparatuses. These apparatuses and components, including but not limited to wound overlays, backing layers, cover layers, drapes, sealing layers, spacer layers, absorbent layers, transmission layers, wound contact layers, packing materials, fillers and/or fluidic connectors are sometimes collectively referred to herein as dressings.

It will be appreciated that throughout this specification reference is made to a wound. It is to be understood that the term wound is to be broadly construed and encompasses open and closed wounds in which skin may be torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sternotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

It will be understood that embodiments of the present disclosure are generally applicable to use in NPWT or topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, 1013.25 mbar, etc.). Accordingly, a negative pressure value of-X mmHg reflects absolute pressure that is X mmHg below 760 mmHg or, in other words, an absolute pressure of (760−X) mmHg. In addition, negative pressure that is “less” or “smaller” than X mmHg corresponds to pressure that is closer to atmospheric pressure (such as, −40 mmHg is less than −60 mmHg). Negative pressure that is “more” or “greater” than −X mmHg corresponds to pressure that is further from atmospheric pressure (such as, −80 mmHg is more than −60 mmHg).

In some cases, local ambient atmospheric pressure is used as a reference point, and such local atmospheric pressure may not necessarily be, for example, 760 mmHg. The negative pressure range can be approximately −80 mmHg, or between about −20 mmHg and −200 mmHg. Note that these pressures are relative to normal ambient atmospheric pressure, which can be 760 mmHg. Thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the negative pressure apparatus.

### Wound Dressing

A source of negative pressure (such as a pump) and some or all other components of the TNP system, such as power source(s), sensor(s), connector(s), user interface component(s) (such as button(s), switch(es), speaker(s), screen(s), etc.) and the like, can be integral with the wound dressing.

The material layers can include a wound contact layer, one or more absorbent layers, one or more transmission or spacer layers, and a backing layer or cover layer covering the one or more absorbent and transmission or spacer layers. The wound dressing can be placed over a wound and sealed to the wound with the pump and/or other electronic components contained under the cover layer within the wound dressing.

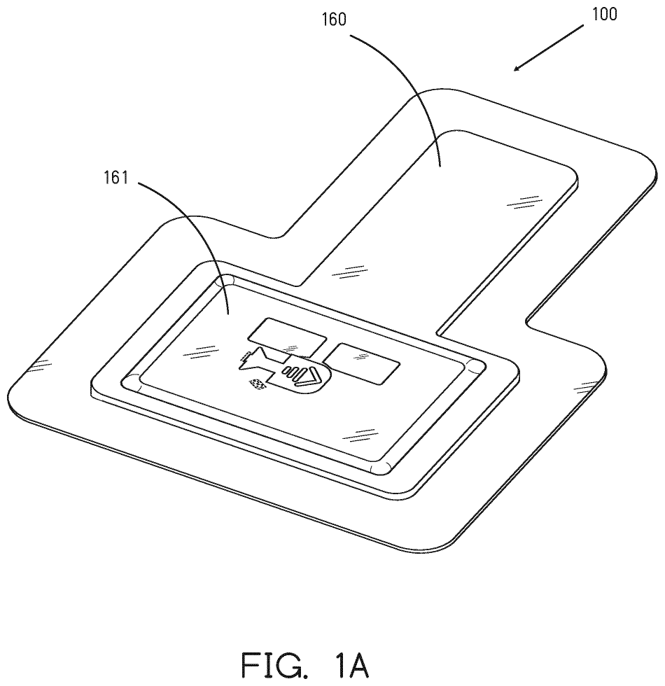

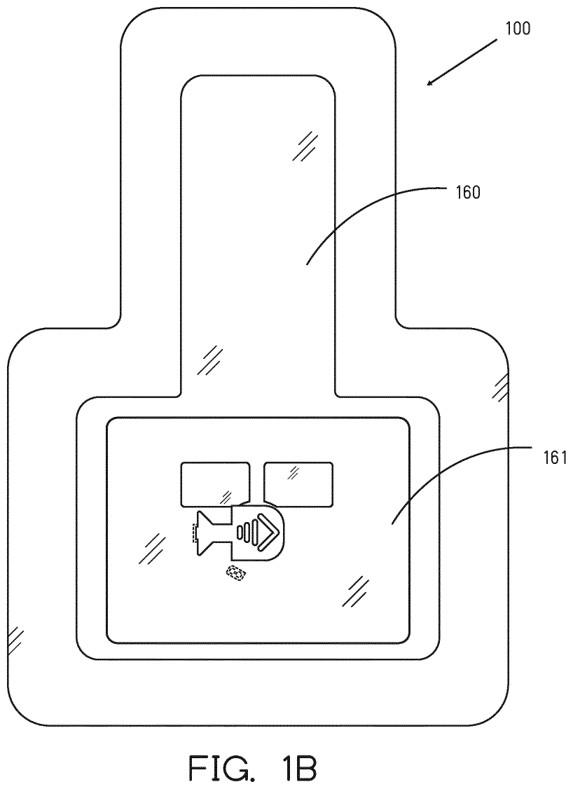

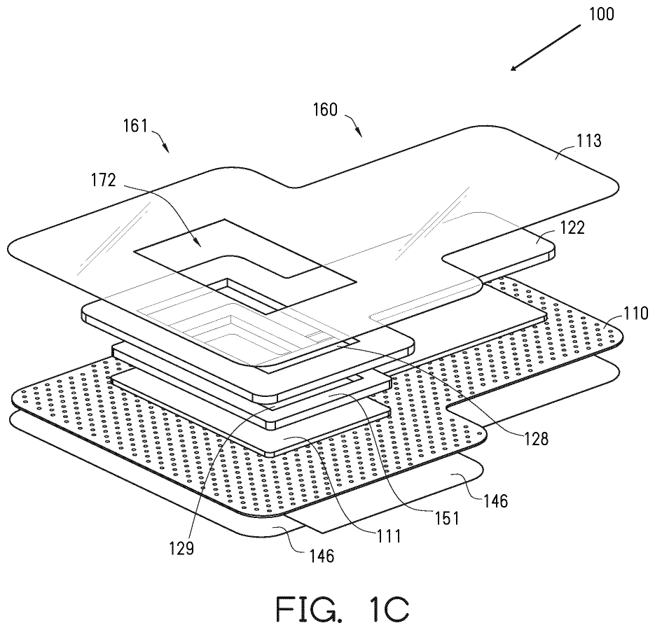

The dressing can be provided as a single article with all wound dressing elements (including the pump) pre-attached and integrated into a single unit. A periphery of the wound contact layer can be attached to the periphery of the cover layer enclosing all wound dressing elements as illustrated in FIG. 1A-1C.

The pump and/or other electronic components can be configured to be positioned adjacent to or next to the absorbent and/or transmission layers so that the pump and/or other electronic components are still part of a single article to be applied to a patient. The pump and/or other electronics can be positioned away from the wound site.

Although certain features disclosed herein may be described as relating to systems and method for controlling operation of a negative pressure wound therapy system in which the pump and/or other electronic components are positioned in or on the wound dressing, the systems and methods disclosed herein are applicable to any negative pressure wound therapy system or any medical device.

FIGS. 1A-1C illustrate a wound dressing incorporating the source of negative pressure and/or other electronic components within the wound dressing. FIGS. 1A-1C illustrate a wound dressing 100 with the pump and/or other electronics positioned away from the wound site. The wound dressing can include an electronics area 161 and an absorbent area 160. The dressing can comprise a wound contact layer 110 (not shown in FIGS. 1A-1B) and a moisture vapor permeable film, cover layer or backing layer 113 positioned above the contact layer and other layers of the dressing. The wound dressing layers and components of the electronics area as well as the absorbent area can be covered by one continuous cover layer 113 as shown in FIGS. 1A-1C.

A layer 111 of porous material can be located above the wound contact layer 110. As used herein, the terms porous material, spacer, and/or transmission layer can be used interchangeably to refer to the layer of material in the dressing configured to distribute negative pressure throughout the wound area. This porous layer, or transmission layer, 111 allows transmission of fluid including liquid and gas away from a wound site into upper layers of the wound dressing. In particular, the transmission layer 111 preferably ensures that an open air channel can be maintained to communicate negative pressure over the wound area even when the absorbent layer has absorbed substantial amounts of exudates. The layer 111 should preferably remain open under the typical pressures that will be applied during negative pressure wound therapy as described above, so that the whole wound site sees an equalized negative pressure. The layer 111 may be formed of a material having a three-dimensional structure. For example, a knitted or woven spacer fabric (for example Baltex 7970 weft knitted polyester) or a non-woven fabric could be used.

Further, one or more absorbent layers (such as layers 122, 151) for absorbing and retaining exudate aspirated from the wound can be utilized. A superabsorbent material can be used in the absorbent layers 122, 151. The one or more layers 122, 151 of absorbent material may be provided above the transmission layer 111. Since in use each of the absorbent layers experiences negative pressures, the material of the absorbent layer can be chosen to absorb liquid under such circumstances. The absorbent layers 122, 151 may comprise a composite comprising superabsorbent powder, fibrous material such as cellulose, and bonding fibers. The composite can be an airlaid, thermally-bonded composite.

The electronics area 161 can include a source of negative pressure (such as a pump) and some or all other components of the TNP system, such as power source(s), sensor(s), connector(s), user interface component(s) (such as button(s), switch(es), speaker(s), screen(s), etc.) and the like, that can be integral with the wound dressing. For example, the electronics area 161 can include a button or switch (shown in FIGS. 1A-1B as being covered by a pull tab). The button or switch can be used for operating the pump (such as, turning the pump on/off).

The electronics area 161 of the dressing can comprise one or more layers of transmission or spacer material and/or absorbent material and electronic components can be embedded within the one or more layers of transmission or spacer material and/or absorbent material. The layers of transmission or absorbent material can have recesses or cut outs to embed the electronic components within whilst providing structure to prevent collapse. As shown in FIG. 1C, recesses 128 and 129 can be provided in absorbent layers 151 and 122, respectively.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound. Additionally, the layers can have a proximal wound-facing face referring to a side or face of the layer closest to the skin or wound and a distal face referring to a side or face of the layer furthest from the skin or wound.

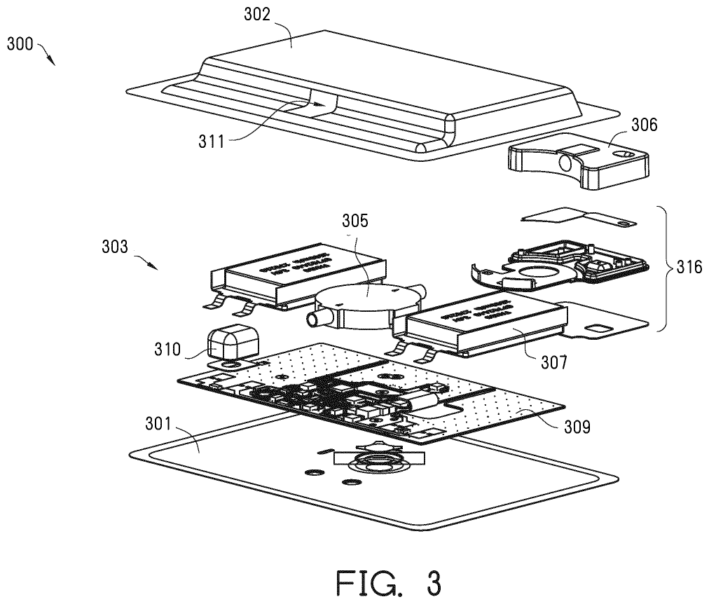

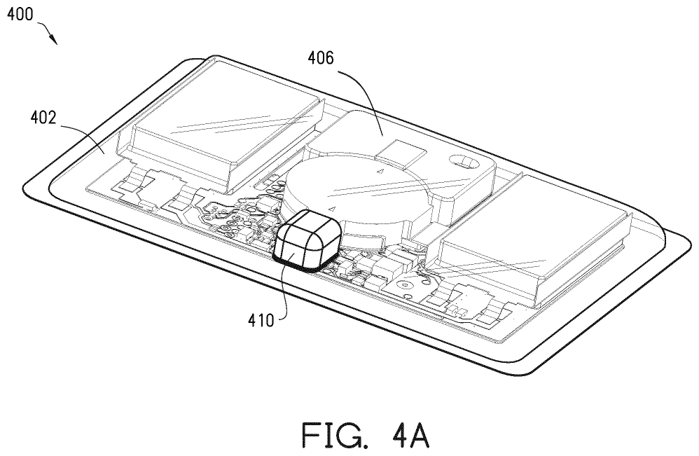

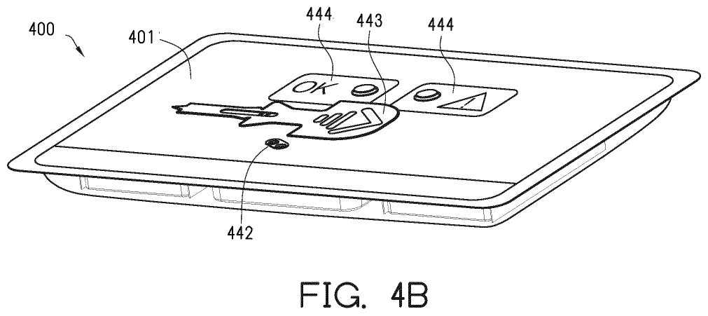

The cover layer may include a cutout 172 positioned over at least a portion of the aperture 128 in the absorbent layer 122 to allow access and fluid communication to at least a portion of the absorbent layers 122 and 151, transmission layer 111, and would contact layer 110 positioned below. An electronics assembly such as described below can be positioned in the apertures 128, 129, and 172 of the first and second absorbent material 151 and 122 and the cover layer 113. The electronics assembly can include a pump, power source, and a printed circuit board as described with reference to FIGS. 3 and 4A-4B.

Before use, the dressing can include one or more delivery layers 146 adhered to the bottom surface of the wound contact layer. The delivery layer 146 can cover adhesive or apertures on the bottom surface of the wound contact layer 110. The delivery layer 146 can provided support for the dressing and can assist in sterile and appropriate placement of the dressing over the wound and skin of the patient. The delivery layer 146 can include handles that can be used by the user to separate the delivery layer 146 from the wound contact layer 110 before applying the dressing to a wound and skin of a patient.

Electronics Assembly Incorporated Within the Wound Dressing

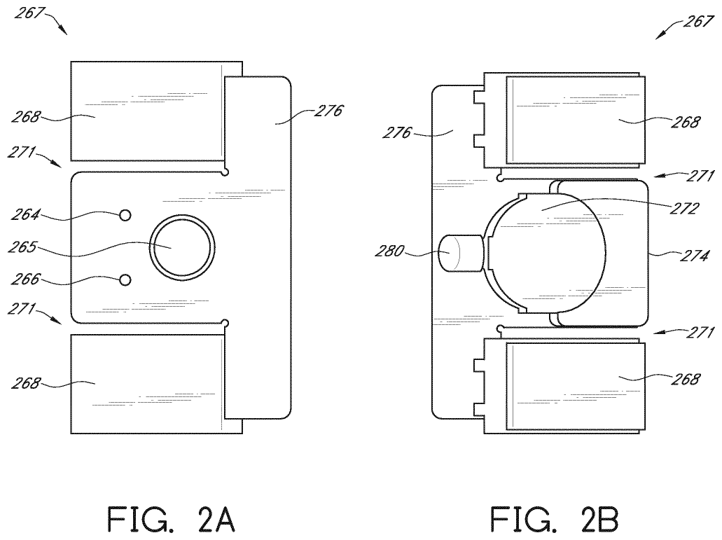

FIGS. 2A-2B illustrate an electronics unit 267 that can be incorporated into a wound dressing. FIG. 2A illustrates the top view of the electronics unit. FIG. 2B illustrates a bottom or wound facing surface of the electronics unit. The electronics unit 267 can include a pump 272 and one or more power sources 268, such as batteries. The electronics unit 267 can include a circuit board 276 configured to be in electrical communication with the pump 272 and/or power source 268. The circuit board 276 can be flexible or substantially flexible.

As illustrated in FIG. 2A, the electronics unit 267 can include single button or switch 265 on the upper surface of the unit. The single button or switch 265 can be used as an on/off button or switch to stop and start operation of the pump and/or electronic components. The electronics unit 267 can also include one or more vents or exhaust apertures 264 on the circuit board 276 for expelling the air exhausted from the pump. As shown in FIG. 2B, a pump outlet exhaust mechanism 274 (sometimes referred to as pump exhaust mechanism or pump outlet mechanism) can be attached to the outlet of the pump 272.

The electronics unit 267 can include a pump inlet protection mechanism 280 as shown in FIG. 2B positioned on the portion of the electronics unit closest to the absorbent area and aligned with the inlet of the pump 272. The pump inlet protection mechanism 280 is positioned between the pump inlet and the absorbent area or absorbent layer of the dressing. The pump inlet protection mechanism 280 (or any of the inlet protection mechanisms disclosed) can include hydrophobic material to prevent fluid from entering the pump 272. The pump inlet protection mechanism 280 (or any of the inlet protection mechanisms disclosed herein) can include a filter.

The upper surface of the electronics unit 267 can include one or more indicators 266 for indicating a condition of the pump and/or level of pressure within the dressing. The indicators can be small light emitting diodes (LED) or other light source that are visible through the dressing components or through holes in the dressing components above the indicators. The indicators can be green, yellow, red, orange, or any other color. For example, there can be two lights, one green light and one orange light. The green light can indicate the device is working properly and the orange light can indicate that there is some issue with the pump (such as, leak, saturation level of the dressing, blockage downstream of the pump, exhaust blockage, low battery, or the like).

The power source 268 can be in electrical communication with the circuit board 276. One or more power source connections are connected to a surface of the circuit board 276. The circuit board 276 can have other electronics incorporated within. For example, the circuit board 276 may support various sensors including, but not limited to, one or more pressure sensors, temperature sensors, optic sensors and/or cameras, and/or saturation indicators.

FIG. 3 illustrates an electronics assembly 300 enclosing an electronics unit 303 within a housing. As illustrated in FIG. 3, the housing of the electronics assembly 300 can include a plate 301 and flexible film 302 enclosing the electronics unit 303 within. The electronics unit 303 can include a pump 305, inlet protection mechanism 310, pump exhaust mechanism 306, power source 307, and circuit board 309. The circuit board 309 can be flexible or substantially flexible.

As is illustrated, the pump exhaust mechanism 306 can be an enclosure, such as a chamber. The electronics unit 303 and pump 305 can be used without the inlet protection mechanism 310. However, the pump exhaust mechanism 306 and the pump 305 can sit within an extended casing 316.

The flexible film 302 can be attached to the plate 301 to form a fluid tight seal and enclosure around the electronic components. The flexible film 302 can be attached to the plate at a perimeter of the plate by heat welding, adhesive bonding, ultrasonic welding, RF welding, or any other attachment or bonding technique.

The flexible film 302 can include an aperture 311. The aperture 311 can allow the inlet protection mechanism 310 to be in fluid communication with the absorbent and/or transmission layers of the wound dressing. The perimeter of the aperture 311 of the flexible film 303 can be sealed or attached to the inlet protection mechanism 310 to form a fluid tight seal and enclosure around the inlet protection mechanism 310 allowing the electronic components 303 to remain protected from fluid within the dressing. The flexible film 302 can be attached to the inlet protection mechanism 310 at a perimeter of the inlet protection mechanism 310 by heat welding, adhesive bonding, ultrasonic welding, RF welding, or any other attachment or bonding technique. The inlet protection mechanism 310 can prevent wound exudate or liquids from the wound and collected in the absorbent area 160 of the wound dressing from entering the pump and/or electronic components of the electronics assembly 300.

The electronics assembly 300 illustrated in FIG. 3 can be incorporated within the wound dressing such that, once the dressing is applied to the body of the patient, air from within the dressing can pass through the inlet protection mechanism 310 to be pumped out toward the pump exhaust mechanism 306 in communication with an aperture in the casing 316 and the circuit board 309 as described herein.

FIGS. 4A-B illustrate an electronics assembly 400 including a pump inlet protection mechanism 410 sealed to the exterior of the flexible film 402, similar to the description with reference to FIG. 3. Also shown is an exhaust mechanism 406, which can be similar to the exhaust mechanism 306.

FIG. 4A illustrates lower, wound facing surface of the electronics assembly 400. FIG. 4B shows an upper surface of the plate 401 (which can face the patient or user) of the electronics assembly 400. The upper surface of the plate 401 can include an on/off switch or button cover 443 (illustrated as a pull tab), indicators 444, and/or one or more vent holes 442. Removal of the pull tab 443 can cause activation of the electronics assembly 400, such as provision of power from the power source to the electronics assembly. Further details of operation of the pull tab 443 are described in PCT International Application No. PCT/EP2018/079745, filed Oct. 30, 2018, titled “SAFE OPERATION OF INTEGRATED NEGATIVE PRESSURE WOUND TREATMENT APPARATUSES,” which is incorporated by reference in its entirety herein.

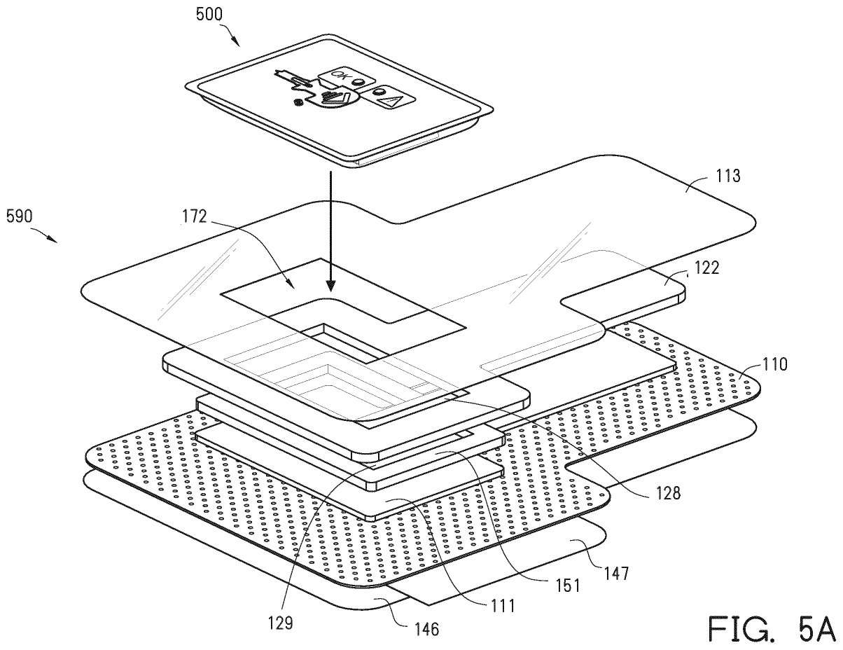

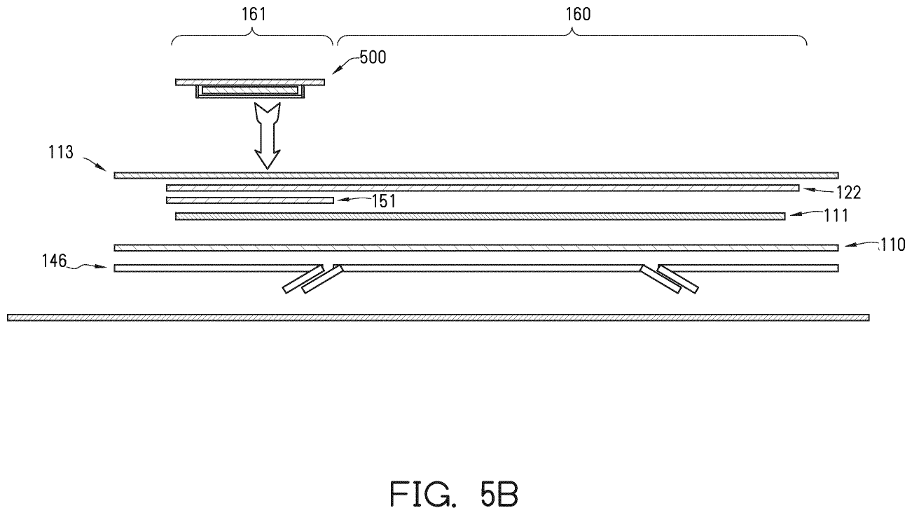

The electronics assembly 400 with the pump inlet protection mechanism 410 extending from and sealed to the film 402 can be positioned within the aperture 172 in the cover layer 113 and absorbent layer(s) (122, 151) as shown in FIG. 1C. The perimeter of the electronics assembly 400 can be sealed to a top surface of the outer perimeter of the aperture 172 in the cover layer 113 as shown in FIGS. 1C and described in more detail with reference to FIG. 5A-5B herein. The electronics assembly 400 can be sealed to the cover layer 113 with a sealant gasket, adhesive, heat welding, adhesive bonding, ultrasonic welding, RF welding, or any other attachment or bonding technique. The electronics assembly 400 can be permanently sealed to the cover layer 113 and could not be removed from the cover layer without destroying the dressing.

The electronics assembly 400 can be utilized in a single dressing and disposed of with the dressing. In some cases, the electronics assembly 400 can be utilized in a series of dressings.

FIG. 5A illustrates a wound dressing, such as the one in FIG. 1C, incorporating an electronics assembly 500 within the wound dressing layers 590. FIG. 5B illustrates a cross-sectional view of the wound dressing incorporating the electronics assembly of FIG. 5A. The electronics assembly 500 can be provided within the aperture 172 in the cover layer and apertures 129 and 128 in the first and second absorbent layers 122, 151. The electronics assembly 500 can seal to the outer perimeter of the aperture 172 of the cover layer. The dressing can comprise a wound contact layer 110 and a moisture vapor permeable film, cover layer or backing layer 113 positioned above the contact layer 110 and other layers of the dressing. A layer 111 of porous material can be located above the wound contact layer 110. As used herein, the terms porous material, spacer, and/or transmission layer can be used interchangeably to refer to the layer of material in the dressing configured to distribute negative pressure throughout the wound area. This porous layer, or transmission layer, 111 allows transmission of fluid including liquid and gas away from a wound site into upper layers of the wound dressing. Further, one or more absorbent layers (such as layers 122, 151) for absorbing and retaining exudate aspirated from the wound can be utilized. The one or more layers 122, 151 of absorbent material may be provided above the transmission layer 111. There may be a small apertured absorbent layer 151 and a large apertured absorbent layer 122. The small apertured absorbent layer 151 can be positioned on top of the large apertured absorbent layer 122. In some cases, the small apertured absorbent layer 151 can be positioned below of the large apertured absorbent layer 122. Before use, the dressing can include one or more delivery layers 146 adhered to the bottom surface of the wound contact layer. The delivery layer 146 can cover adhesive or apertures on the bottom surface of the wound contact layer 110.

FIG. 6 illustrate another configuration of an electronics assembly 600 similar to the configurations of the electronics assembly 300, 400, 500 illustrated in and described in relation to FIGS. 3-5B. Reference numerals of the same or substantially the same features may share the same last two digits. In some configurations, the electronics unit 603 of the electronics assembly 600 can be similar to the configuration of the electronic unit 267 illustrated in and described in relation to FIGS. 2A-2B.

The electronics assembly 600 can include an electronics unit 603 at least partially enclosed within a housing. For example, the housing can include a plurality of flexible film layers 602a, 602b. In the illustrated configuration, the plurality of flexible film layers 602a, 602b includes a first or bottom flexible film layer 602a and a second or top flexible film layer 602b. The first and second flexible film layers 602a, 602b can be attached to form a fluid tight seal and enclosure around the electronic components. For example, the flexible film layers 602a, 602b can be attached at a perimeter of the flexible film layers 602a, 602b by heat welding, adhesive bonding, ultrasonic welding, RF welding, or any other attachment or bonding technique. The plurality of flexible film layers 602a, 602b can include a polymeric material, such as polyurethane, a paper material, or any other suitable material. In some cases, the flexible film layers 602a, 602b can be a waterproof and/or gas tight film material. In some cases, the flexible film layers 602a, 602b can be made of a polyurethane material (such as a thermoplastic polyurethane), polyester, ethylene-vinyl acetate, polyethylene, and/or any other suitable material. Advantageously, the plurality of flexible film layers 602a, 602b can enable the dressing to be flexible and conformable while protecting the electronic unit 603 in use. In some configurations, the plurality of flexible film layers 602a, 602b can be configured to prevent condensation in the volume within the plurality of flexible film layers 602a, 602b. For example, the plurality of flexible film layers 602a, 602b can include material with a moisture vapor permeability rating (MVP) such that vapor pass through the plurality of flexible film layers 602a, 602b.

The housing of the electronics assembly can also include an upper housing portion or label 601. The upper housing portion can include a material layer forming a label for the electronics assembly. As used herein the terms label or plate (such as label 601 and plate 301, 401) can be used interchangeable with the upper housing portion and the terms label and plate refer to the same or similar portions of the electronics assembly that form an upper housing portion providing the upper most component or the layer or component furthest from the wound when the electronics assembly within the dressing is positioned over the wound. As shown in the exploded perspective view of FIG. 6, the upper housing portion or label 601 can include indicia, windows, embossed features, or any other components that provide a user interface or other features that communicate information to the user or allow the user to communicate with the electronics of the electronic assembly.

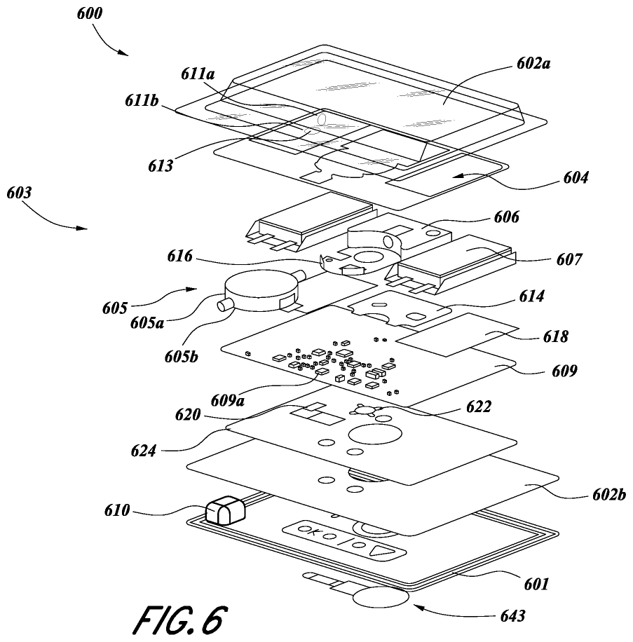

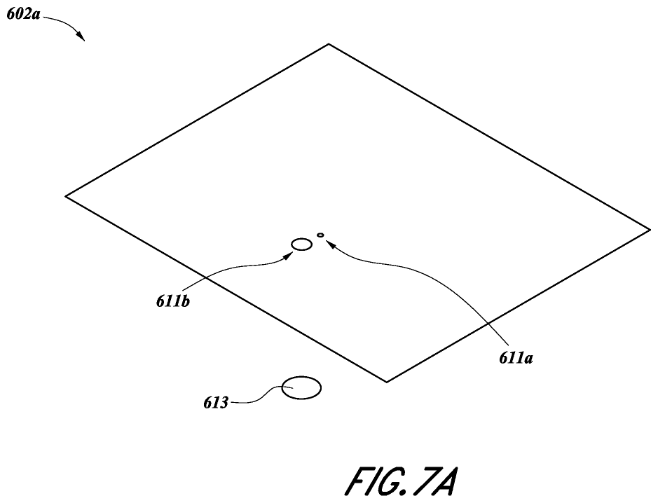

As shown in FIGS. 7A-7B, the first flexible film layer 602a can include one or more apertures 611a, 611b. In the illustrated configuration, the one or more apertures 611a, 611b includes a first aperture 611a and a second aperture 611b. The first aperture 611a can allow the pump 605 to be in fluid communication with the absorbent and/or transmission layers of the wound dressing. For example, the pump 605 can include a body portion 605a and an inlet 605b extending from the body portion 605a as shown in FIG. 6. The inlet 605b can extend through the first aperture 611a such that the inlet 605b can be external to the first flexible film layer 602a while the body portion 605a can be enclosed within the first and second flexible film layer 602a, 602b. In some configurations, the inlet 605b can include a nozzle 605b that can be tapered from the body portion 605a to an end of the nozzle 605b. For example, the portion of the nozzle 605b adjacent the body portion 605a (i.e., a base portion of the nozzle 605b) can have a larger diameter than the end of the nozzle 605b. The first aperture 611a can be configured to form a hermetic and/or fluid tight seal around the nozzle 605b to prevent wound exudate from entering into the volume within the first and second flexible film layers 602a, 602b. For example, as shown in FIGS. 7A-7B, the first aperture 611a can include a diameter smaller than the larger diameter of the base portion of the nozzle 605b such that the first aperture 611a stretches to form the seal around the nozzle 605b. This seal can protect the electronic unit 603 fluid within the dressing.

In some configurations, the second aperture 611b can be positioned above the inlet 605b (i.e., further from or opposite side from the wound bed) when the electronics assembly 600 is assembled with the dressing. The circuit board 609 can support various sensors including, but not limited to, one or more pressure sensors, temperature sensors, optic sensors and/or cameras, and/or saturation indicators. For example, as shown in FIG. 6, the circuit board 609 can include a pressure sensor 609a. The second aperture 611b can be aligned with the pressure sensor 609a so that the pressure sensor 609a can measure the pressure from the inlet 605b in use. In some configurations, the electronics unit 604 can include a first pressure sensor 609a and a second pressure sensor (not shown). In this alternative configuration, the second pressure sensor can be positioned outside the first flexible film layer 602a and configured to measure a pressure at the wound site. As shown in FIGS. 6 and 7A-7B, in some configurations, the filter 613 can be positioned between the second aperture 611b and the pressure sensor 609a. For example, the filter 613 can be adhered to the first flexible film layer 602a. In other configurations, the second aperture 611b can be configured to receive a filter 613. The filter 613 can be configured to prevent particulates, wound exudate, and/or fluid from coming into contact with the pressure sensor 609a.

The inlet protection mechanism 610 can prevent wound exudate or liquids from the wound and collected in the absorbent area of the wound dressing from entering the pump 605 and/or electronic unit 603 of the electronics assembly 600. The pump inlet protection mechanism 610 can include hydrophobic material to prevent fluid from entering the pump 605. In some configurations, the inlet protection mechanism 610 can removably couple to the inlet 605b. For example, the inlet protection mechanism 610 can couple to the inlet 605b via a mechanical fit (e.g., a push fit, a friction fit). The inlet protection mechanism 610 can be configured to form a fluid tight seal over the inlet 605b via the mechanical fit and without an adhesive. In other embodiments, the inlet protection mechanism can be adhered to or attached to the inlet 605b. For example, the inlet protection mechanism can be adhered to the inlet 605b with glue or other adhesive. The inlet protection mechanism 610 can be secured to the first flexible film layer 602a and/or the pump 605 via other mechanisms including, but not limited, an adhesive (a double-sided adhesive, a hot melt adhesive, a screen printed adhesive, or glue). The inlet protection mechanism 610 can be positioned external to the volume within the first and second flexible film layers 602a, 602b when the inlet protection mechanism 610 is coupled to the inlet 605b. In some configurations, the inlet protection mechanism 610 can enclose the aperture 611b and/or the filter 613 when the inlet protection mechanism 610 is coupled to the inlet 605b.

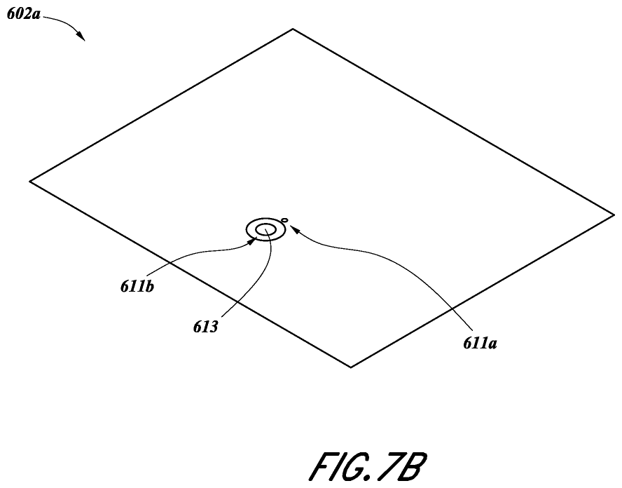

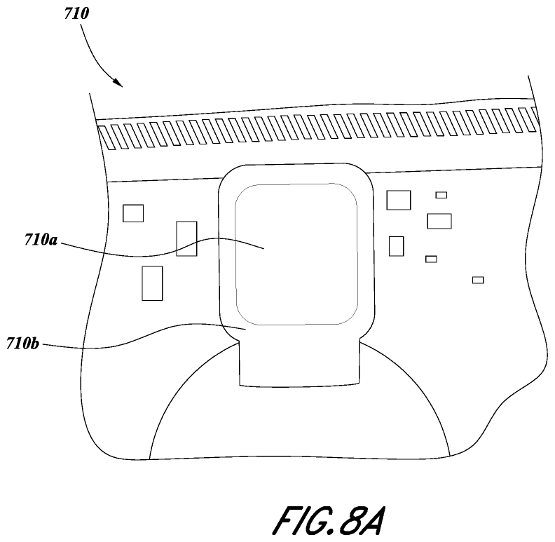

FIGS. 8A-8B illustrate another configuration of an inlet protection mechanism 710 similar to the configurations of the inlet protection mechanism 310, 410, 610 illustrated in and described in relation to FIGS. 3-4A and 6. Reference numerals of the same or substantially the same features may share the same last two digits. The inlet protection mechanism 710 can include a first portion 710a and a second portion 710b. The first portion 710a can include an inner filter element 710a. The second portion 710b can include an outer cap 710b. In the illustrated configuration, the inner filter element 710a can be positioned over the second aperture 611b of the first flexible film layer 602a and attached to the pump 605. The outer cap 710b can be positioned over the inner filter element 710a. The outer cap 710b can be secured to the first flexible film layer 602a via an adhesive (e.g., a double-sided adhesive, a hot melt adhesive, a screen printed adhesive, or glue). The outer cap 710b can comprise of a different material or different materials than the inner filter element 710a. For example, the inner filter element 710a may comprise hydrophobic material(s) such that adhesive may not adhere to the inner filter element 710a. Thus, the outer cap 710b can include a material or materials configured to adhere to the first flexible film layer 602a. By making the outer cap 710b from material(s) that can adhere to an adhesive, the outer cap 710b can be configured to secure the inner filter element 710a to the pump 605 and the first flexible film layer 602a can be adhered to the outer cap 720b such that a fluid tight seal is formed around the outer cap 720b preventing fluid from entering the electronics assembly 600.

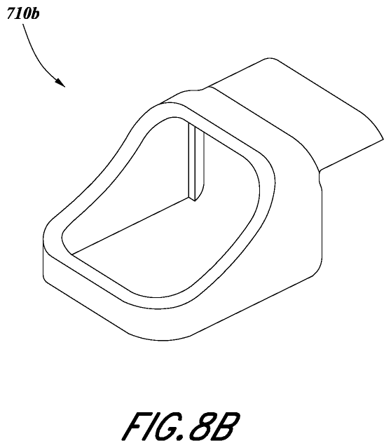

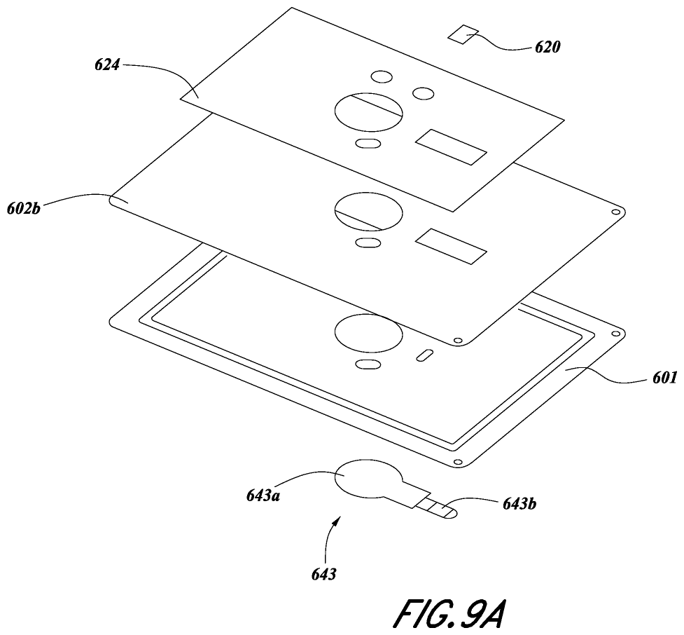

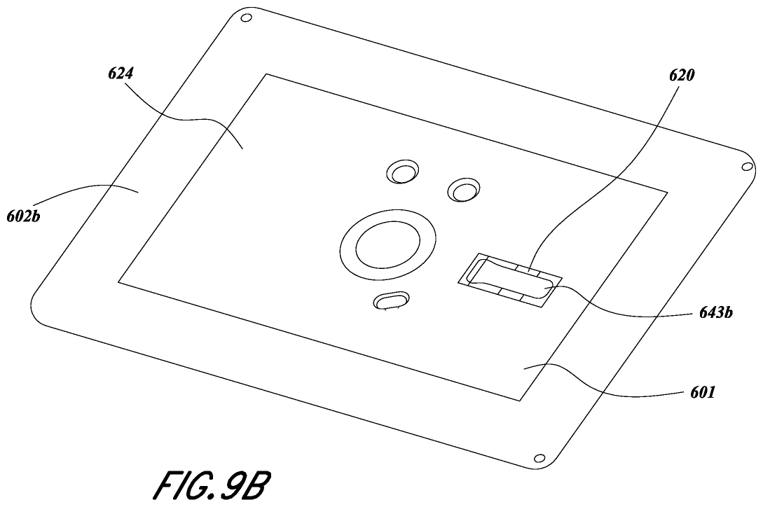

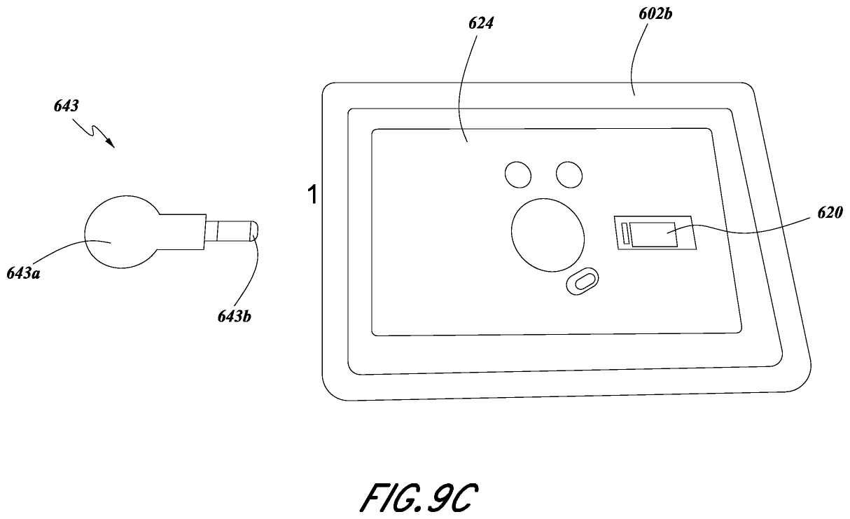

As shown in FIG. 6, the electronics assembly 600 can include a conformal coating 604, a shim 620 (described further below in reference to FIGS. 9A-9H), a button 622, a plurality of gaskets 614, 618, 624. The conformal coating 604 can be positioned within the plurality of flexible film layers 602a, 602b and comprise a plurality of openings configured to receive the one or more power sources 607, the pump exhaust mechanism 606 and/or the pump 605. The conformal coating 604 can be configured to provide mechanical support to the other components of the electronics assembly 600 and retain the one or more power sources 607, the pump exhaust mechanism 606 and/or the pump 605 in place. In some configurations, the conformal coating 604 can be configured to be conformable, act as a heat sink, and/or be resistant to moisture. In some configurations, the conformal coating 604 can cover the entire perimeter of the circuit board 609. In some configurations, the conformal coating 604 can be thicker along an outer edge of the conformal coating 604 to prevent or reduce air and/or liquid ingress. As illustrated in FIG. 6, the button 622 can be used as an on/off button or switch to stop and start operation of the pump 605 and/or electronics unit 603. As shown in FIG. 11, the button 622 can be received by one or apertures of the second flexible film layer 602b and/or the label gasket 624 such that the user can activate the electronics unit 603 by pushing the button 622.

As shown in FIG. 6, the plurality of gaskets 614, 618, 624 can include a pump exhaust gasket 614, one or more power source gaskets 618, and/or a label gasket 624. The gaskets 614, 618, 624 can be a double-sided adhesive. The plurality of gaskets 614, 618, 624 can be configured to secure the different components of the electronics assembly 600 to the circuit board 609. For example, the pump exhaust gasket 614 can be configured to adhere the pump exhaust mechanism 606 to the circuit board 609. The one or more power source gaskets 618 can be configured to adhere the one or more power sources 607 to the circuit board 609. The label gasket 624 can be configured to adhere the second flexible film layer 602b to the circuit board 609.

FIGS. 9A-9E illustrate different views of the label 601, the second flexible film layer 602b, the shim 620, the label gasket 624, and the on/off switch or button cover 643 (illustrated as a pull tab). FIG. 9A illustrates an expanded view of the label 601, the second flexible film layer 602b, the label gasket 624, and the pull tab 643. FIG. 9B illustrates the label 601, the second flexible film layer 602b, the label gasket 624, and the pull tab 643 in an assembled form. In some configurations the label 601 can be bonded to the second flexible film layer 602b by heat bonding, double sided adhesive, PU dispersion and heat bonding, hot melt adhesive, screen printed adhesive, an adhesive or polymer dispersion mixed into the ink used for the label artwork, or any suitable method. In some configurations, the label 601 can include surface additives to enhance the bonding between the second flexible film layer 602b and the label 601. For example, the entire lower surface of the label 601 can be bonded to the second flexible film layer 602b by treating the label 601 with plasma (e.g., oxygen or atmospheric) prior to applying heat against the label 601 and the second flexible film layer 602b. Advantageously, the plasma treatment of the label 601 may increase the bond strength of the bond between the label 601 and the second flexible film layer 602b.

FIGS. 9C and 9D illustrate a bottom view and a top view, respectively, of the pull tab 643 separate from an assembly of the label 601, the second flexible film layer 602b, the shim 620, and the label gasket 624. The pull tab 643 can include a user interface portion 643a and an activation portion 643b. For example, when the pull tab 643 is pulled via the user interface portion 643a, the activation portion 643b can be pushed against the circuit board 609, thereby activating the electronics assembly 600. In some configurations, the pull tab 643 can be positioned between the shim 620 and the circuit board 609 such that the shim 620 can push the activation portion 643b of the pull tab 643 against the circuit board 609 after the pull tab 643 is pulled. For example, the shim 620 can ensure the activation portion 643b of the pull tab 643 contacts the circuit board 609 after the pull tab 643 is pulled. In some configurations, the shim 620 can be the same or similar thickness as the second flexible film layer 602b and the label gasket 624. For example, the shim 620 can include the same material as the second flexible film layer 602b and/or the label gasket 624.

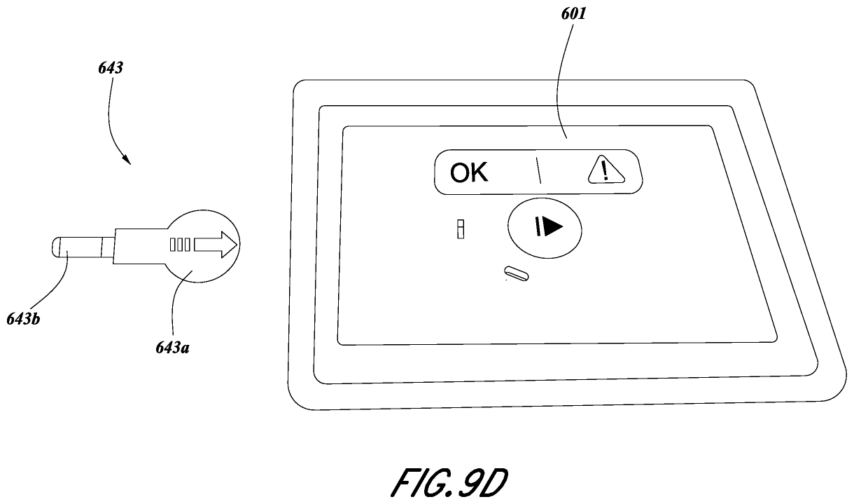

FIG. 10 illustrates another configuration of a label 1101 similar to the configuration of the label 601 illustrated in and described in relation to FIGS. 9A-9E. Reference numerals of the same or substantially the same features may share the same last two digits. The label 1101 can include at least one embossed portion 1101a. The embossed portion 1101a can extend toward the interior of the electronics assembly 600 (e.g., toward second flexible film layer 601b). The embossed portion 1101a can be configured to push the activation portion 643b of the pull tab 643 against the circuit board 609 after the pull tab 643 is pulled. For example, the embossed portion 1101a can ensure the activation portion 643b of the pull tab 643 contacts the circuit board 609 after the pull tab 643 is pulled.

As shown in FIG. 11, the electronics assembly 600 include one or more indicators 644. The one or more indicators 644 can be the same or similar to any of the indicators 266, 444 described herein. As previously described, the one or more indicators 644 can be positioned on an upper surface of the electronics unit 603. For example, the one or more indicators 644 can be positioned on an upper surface of the circuit board 609. In some configurations, the circuit board 609 can include one or more holes configured to receive the one or more indicators 644. When the one or more indicators 644 are positioned in the one or more holes of the circuit board 609, an adhesive (e.g., a light adhesive) can be applied to the one or more holes to prevent from leaking through the one or more holes in use.

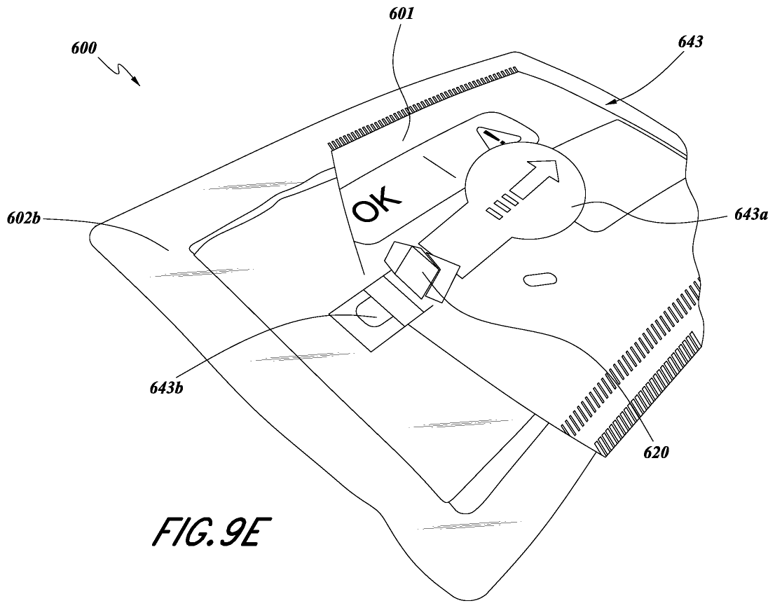

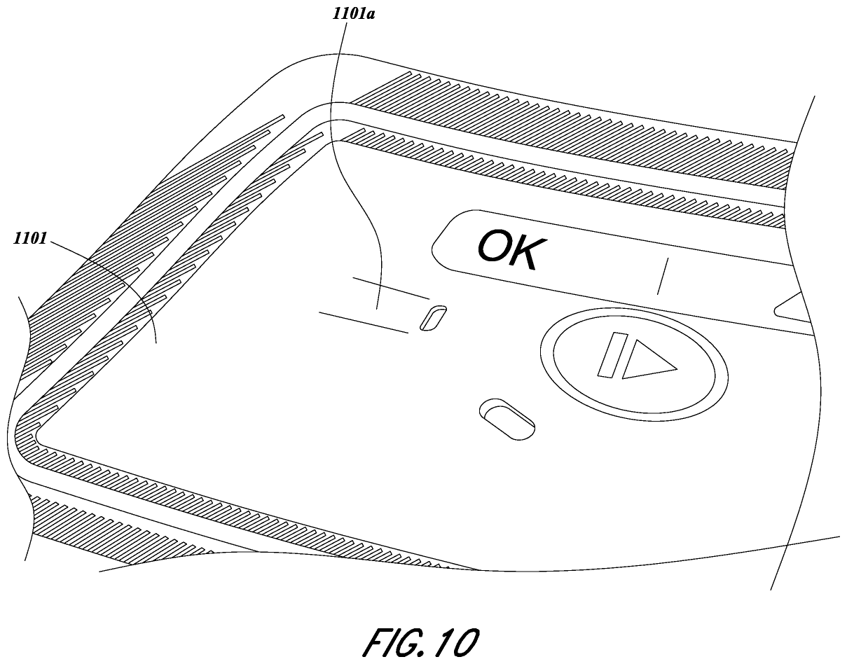

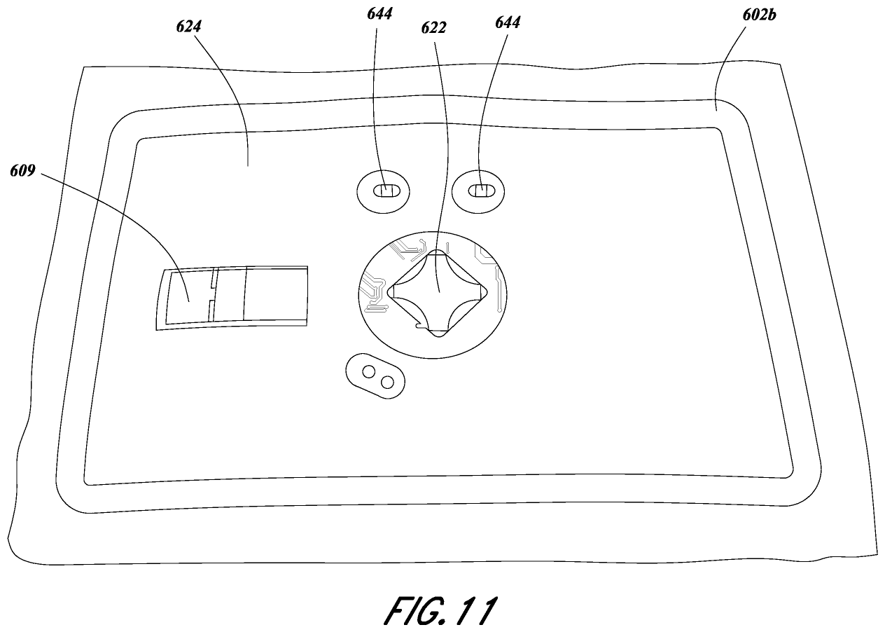

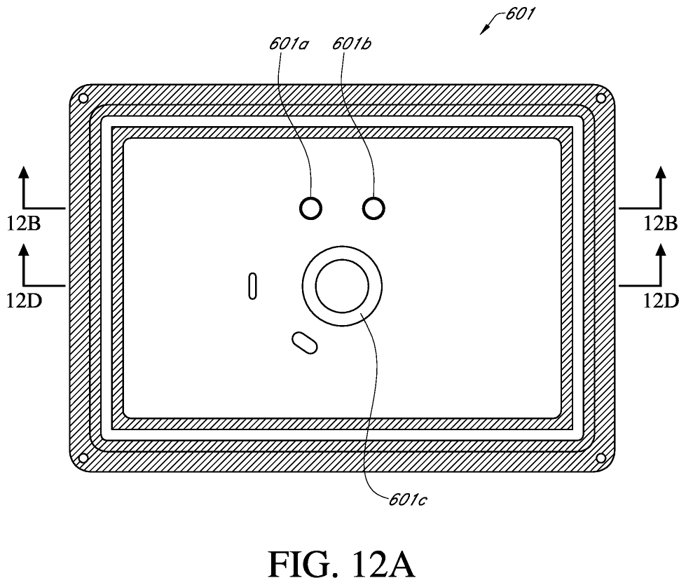

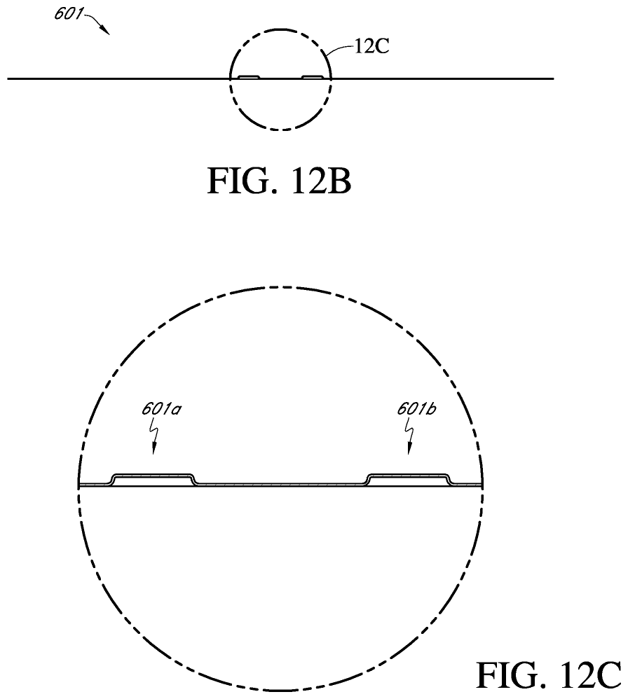

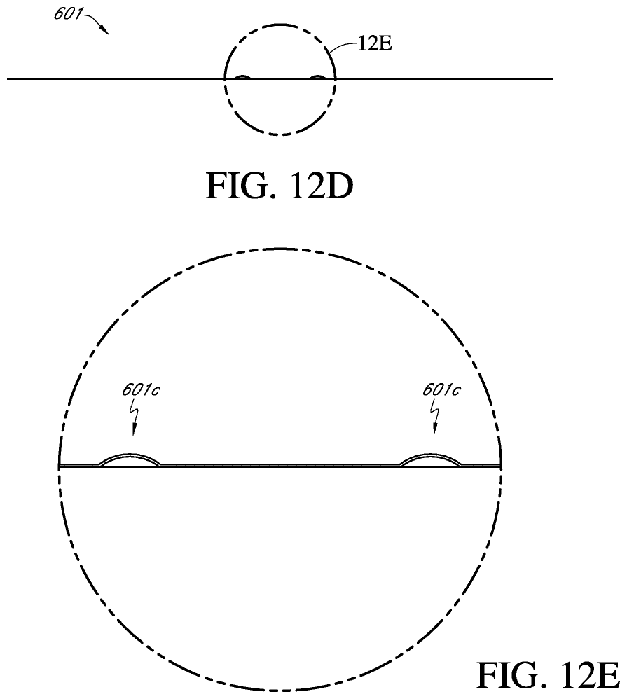

FIGS. 12A-12E illustrate different views of an unprinted label 601. The label 601 can include a plurality of embossed portions 601a, 601b, 601c. The plurality of embossed portions 601a, 601b, 601c can extend away from the second flexible film layer 602b when the electronics assembly 600 is assembled. In some configurations, the plurality of embossed portions can include a first embossed portion 601a, a second embossed portion 601b, and a third embossed portion 601c. FIGS. 12B-12C illustrate cross-sectional views of the first and second embossed portions 601a, 601b. The first and second embossed portions 601a, 601b can align with the one or more indicators (e.g., LED lights or other light source) of the circuit board 609. FIGS. 12D-12E illustrate cross-sectional views of the third embossed portion 601c. The third embossed portion 601c can form a circle around a non-embossed portion such that the on/off button 622 aligns with a central non-embossed portion. For example, the third embossed portion 601c can form a raised ring around the non-embossed portion.

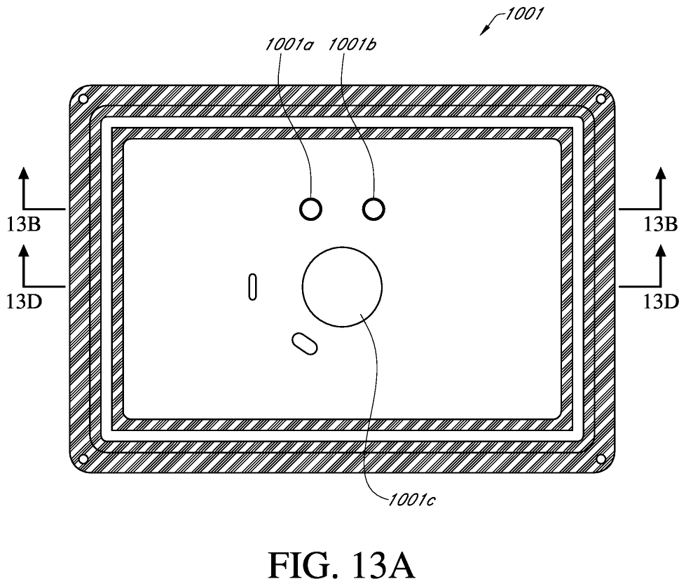

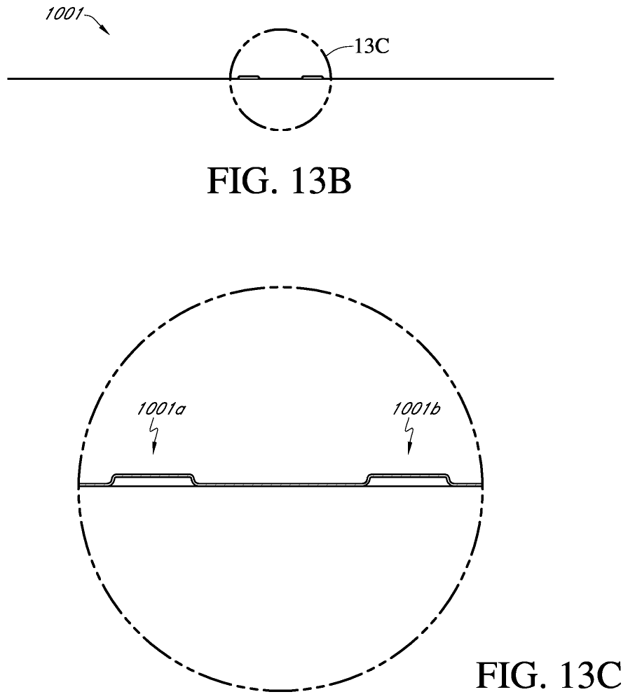

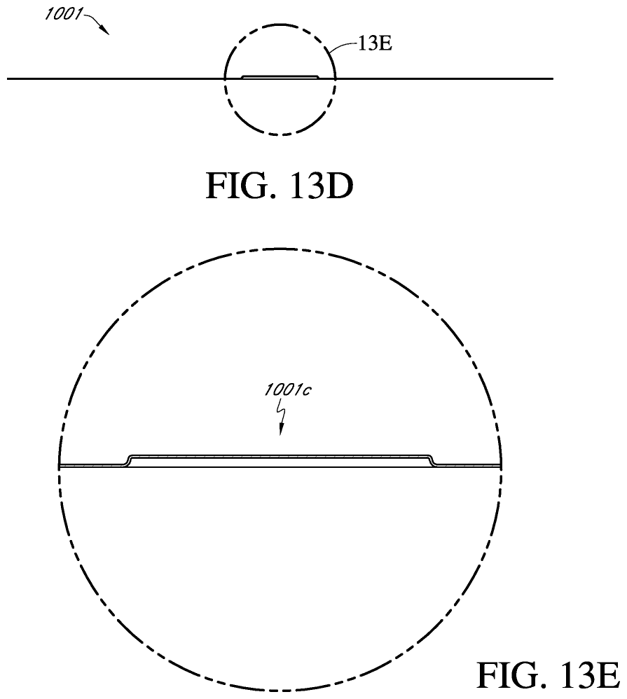

FIGS. 13A-13E illustrate different views of another configuration of a label 1001 similar to the configuration of the label 601 illustrated in and described in relation to FIGS. 12A-12E. Reference numerals of the same or substantially the same features may share the same last two digits. FIGS. 13B-13C illustrate cross-sectional views of the first and second embossed portions 1001a, 1001b. FIGS. 13D-13E illustrate cross-sectional views of the third embossed portion 1001c. The third embossed portion 1001c can include a circular shape or any shape (e.g., rectangular, triangular) that can align with the on/off button 622. For example, the third embossed portion 1001c can include a fully raised shape (e.g., a circle) aligned with the on/off button 622. Advantageously, the third embossed portion 1001c can be configured such that the label 1001 returns to the label's 1001 original position after the user pushes the on/off button 622 via the label 1001.

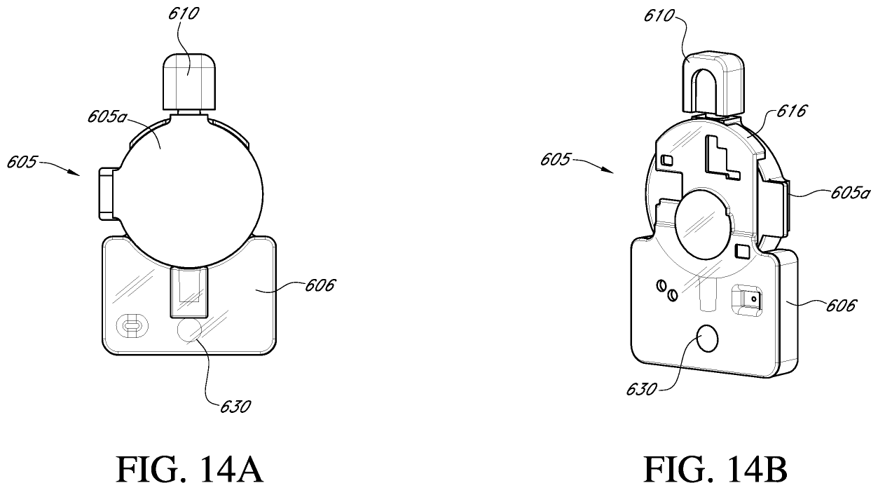

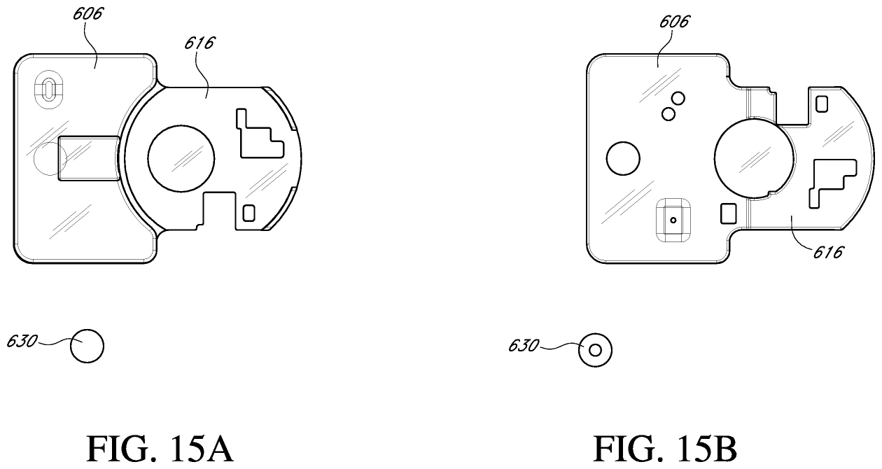

FIGS. 14A-15B illustrate various views of the pump exhaust mechanism 606. For example, the pump exhaust mechanism 606 can include an umbrella valve 630. As previously described, once the dressing is applied to the body of the patient, air from within the dressing can pass through the inlet protection mechanism 610 to be pumped out toward the pump exhaust mechanism 606 in communication with an aperture in the extended casing 616 and the circuit board 609. The pump exhaust mechanism 606 can include one or more valves 630 configured to prevent or reduce the air from leaking through the pump exhaust mechanism 606 toward the dressing. For example, the pump exhaust mechanism 606 can include an umbrella valve 630. In some cases, the one or more valves 630 can be positioned at the vent holes or exhaust holes of the pump exhaust mechanism thereby covering or closing the vent or exhaust opening in the pump exhaust mechanism. The one or more valves 630 can be a one-way valve that prevents air from atmosphere from leaking through the pump exhaust mechanism toward the dressing but can allow air to exit the pump exhaust mechanism to the atmosphere.

The one or more valves 630 can be configured to increase the efficiency of the pump 605. In some configurations, the one or more valves 630 can be configured to reduce or eliminate any whistling or other noise created by the air flowing through the pump exhaust mechanism 606. In some configurations, the one or more valves 630 can be configured to produce a noise. For example, the noise could alert the user that there is a leak in the pump exhaust mechanism 606.

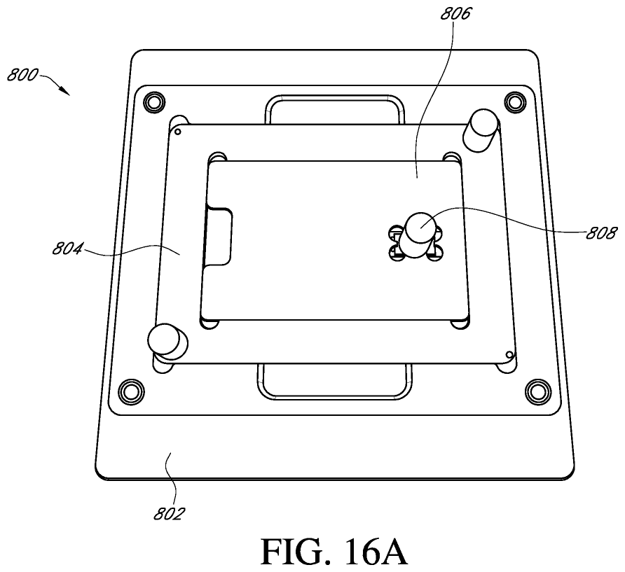

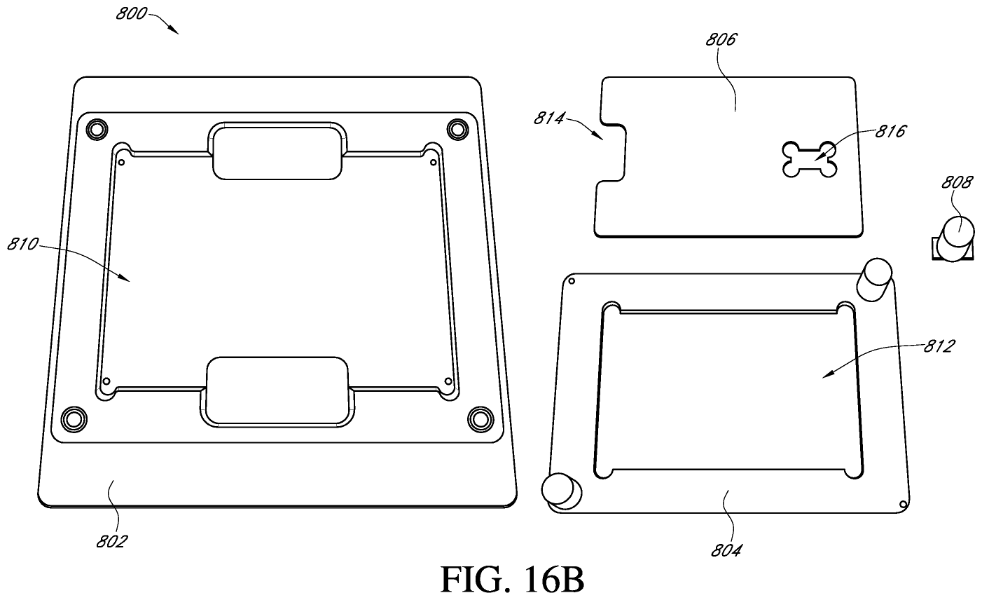

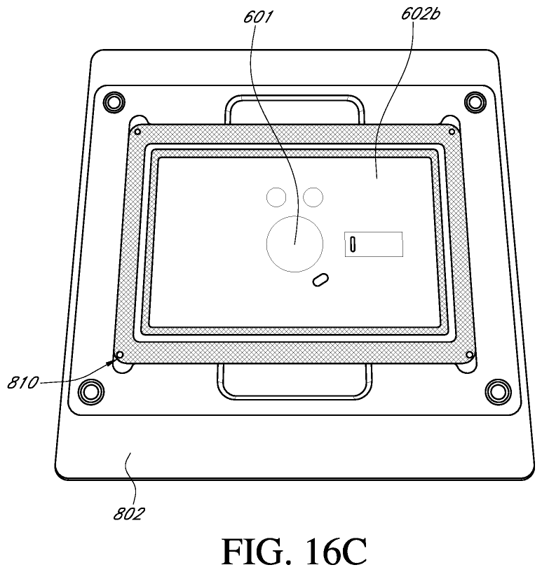

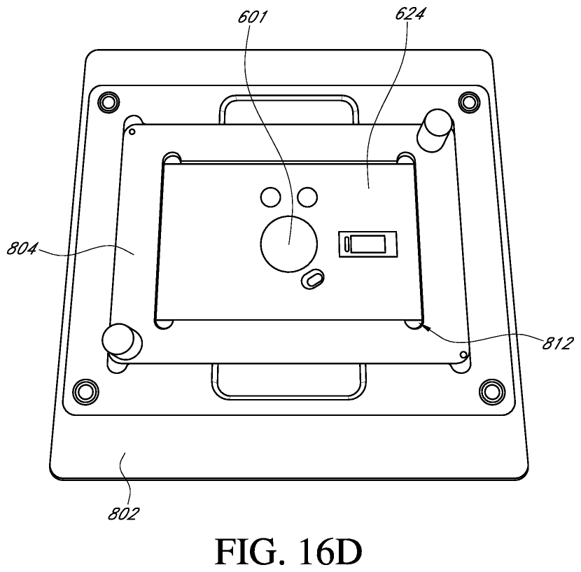

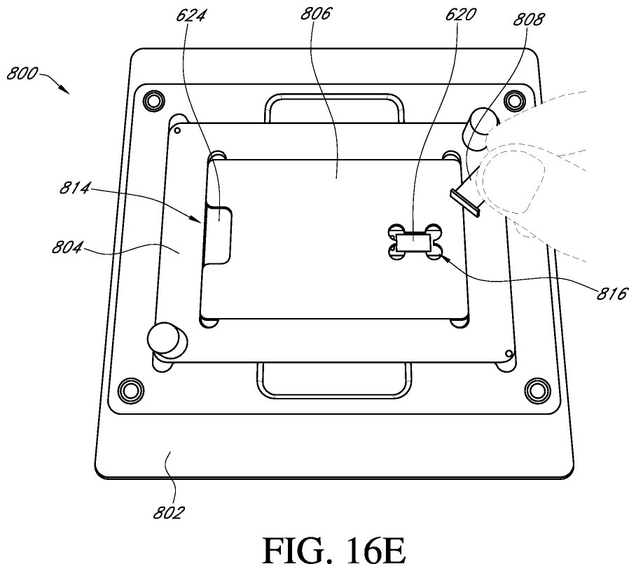

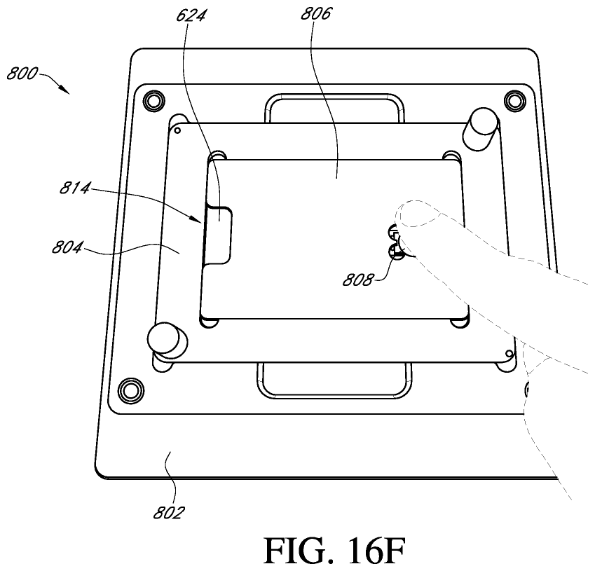

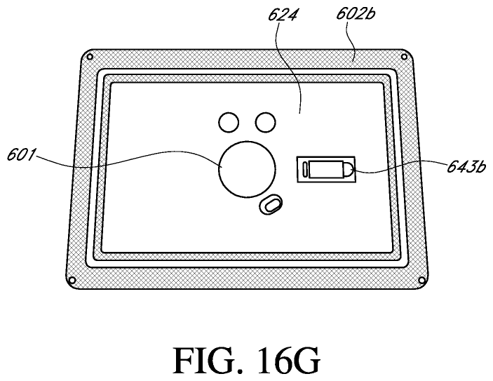

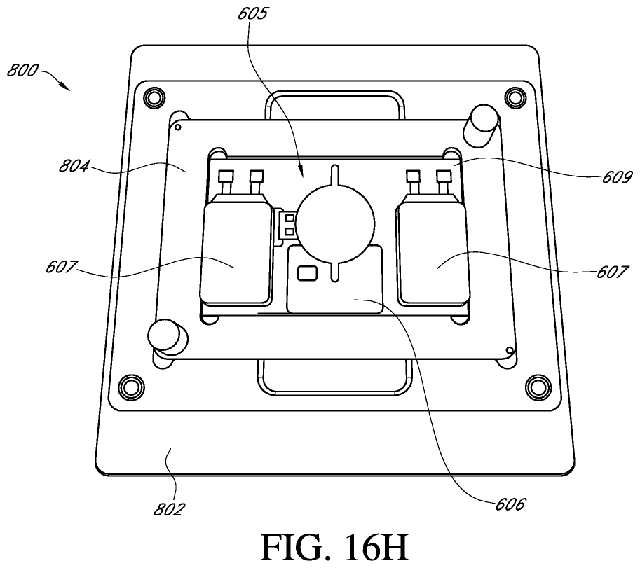

FIGS. 16A-16H illustrate a portion of the process for manufacturing the electronics assembly 600. FIGS. 16A-16B illustrates an alignment jig 800 used to assemble the electronics assembly 600. The alignment jig 800 can include a base 802, a first alignment component 804, a second alignment component 806, and a third alignment component 808. The base 802 can include a central recess 810 configured to receive components of the electronics assembly 600, the first alignment component 804, and the second alignment component 806. The first alignment component 804 can include a central aperture 812 configured to receive the second alignment component 806. The second alignment component 806 can include a first aperture 814 and a second aperture 816. The second aperture 816 can be configured to receive the third alignment component 808.

As shown in FIG. 16C, the label 601 bonded to the second flexible film layer 602b can be placed into the central recess 810 of the base 802. As shown in FIG. 16D, the label gasket 624 can be placed over the second flexible film layer 602b and the first alignment component 804 can be positioned over the label gasket 624. As shown in FIG. 16E, the second alignment component 806 can be positioned over the label gasket 624 and the shim 620 can be positioned through the second aperture 816 of the second alignment feature 806. As shown in FIGS. 16E-16F, the third alignment component 808 can be positioned within the second aperture 816 of the second alignment feature 806 and over the shim 620. As shown in FIGS. 16G, the label assembly, which can include the label 601, the second flexible film layer 602b, the label gasket 624, and the shim 620, can be removed from the alignment jig 800. In some configurations, the label assembly including the label 601, the second flexible film layer 602b, the label gasket 624, and the shim 620 can remain within the alignment jig 800 and the pull tab 643 can be inserted into the label assembly. The pull tab 643 can be inserted through a hole in the label assembly until the activation portion 643b is positioned under the shim 620.

As shown in FIG. 16H, the label assembly with the shim 620 can be reinserted into the base 802 and the circuit board 609 can be positioned on the label gasket 624. The pump 605, the pump exhaust mechanism 606, and/or the power source(s) 607 can be attached to the circuit board 609. In some configurations, the conformal coating 604 (not shown) can be positioned over the circuit board 609 and into the base 802. The first alignment component 804 can be positioned over the circuit board 609 to adhere the circuit board 609 to the label gasket 624. In some configurations, the first alignment component 804 can be removed and the conformal coating 604 (not shown) can be positioned over the circuit board 609.

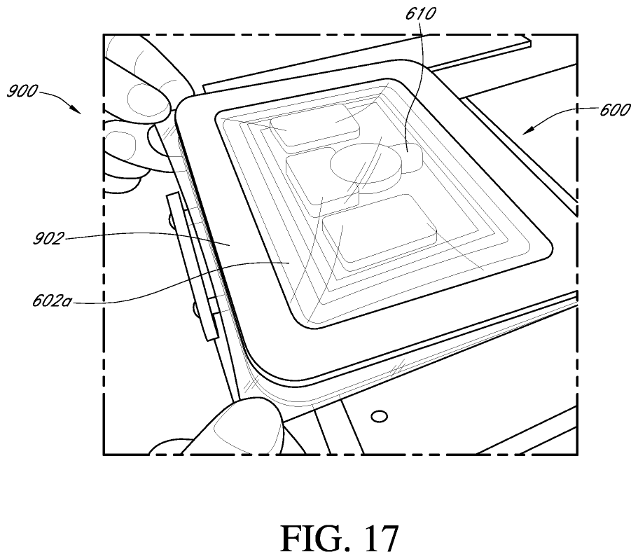

FIG. 17 illustrate another portion of the process for manufacturing the electronics assembly 600. The electronics assembly 600 can be positioned into a sealing tool 900 with the inlet 605b of the pump 605 being positioned through the first aperture 611a of the first flexible film layer 602a and the inlet protection mechanism 610 being positioned on the inlet 605b. A retention ring 902 can be positioned over the first flexible film layer 602a. The retention ring 902 can be configured to minimize creasing of the first flexible film layer 602a when the first flexible film layer 602a is bonded to the second flexible film layer 602b (not shown). The retention ring 902 can be configured to allow the edges of the first flexible film layer 602a to be repositioned to, for example, remove spots of potential creasing during bonding without having to reposition the entire first flexible film layer 602a. In some configurations, a nylon sheet can be placed over the electronics assembly 800 to further reduce creasing during bonding. Advantageously, bonding the first and second flexible film layers 602a, 602b can provide mechanical stability to the enclosed electronics unit 603. As previously discussed, the first and second flexible film layers 602a, 602b can be bonded through any means including, but not limited to, double sided adhesive, hot melt adhesive, screen printed adhesive, or any suitable bonding means.

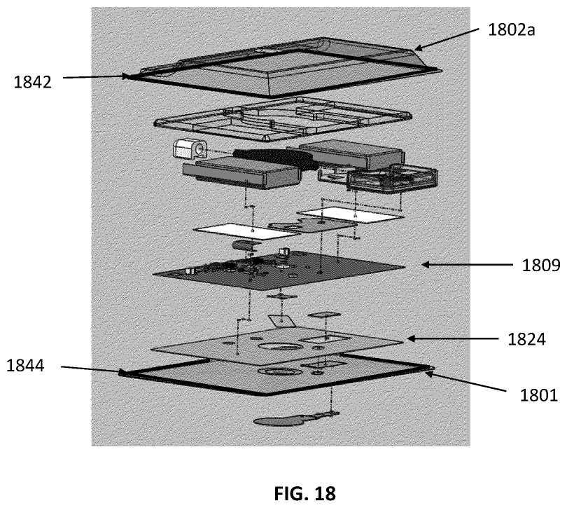

As described herein the electronic assembly 600 can be assembled and sealed in a way to not allow liquid to enter the interior of the electronic assembly 600 to protect the electronics and the printed circuit board within the electronic assembly 600. FIG. 18 illustrates an electronics assembly 1800 similar to the electronics assembly 600 shown in FIG. 6. Reference numerals of the same or substantially the same features may share the same last two digits. However, the housing of the electronics assembly 1800 as shown in FIG. 18 does not include the second flexible film layer provided on the lower surface or wound facing surface of the label 1801. As described with reference to FIG. 6, the label (such as label 1801, 2001, 2101, and 2201) as used herein refers to an upper housing portion of the housing of the electronics assembly. As described herein, the lower surface of a layer or component is the wound facing surface when the electronics assembly is positioned in the dressing over a wound. In some cases, as shown in FIG. 18, the label gasket 1824 can be in contact with the lower surface of the label 1801 when the electronics assembly is assembled. In other cases, where the label gasket 1824 is not used the circuit board 1809 can be in contact with the lower surface of the label when the electronics assembly is assembled. In some cases, when the label gasket 1824 is used, the circuit board 1809 my extend beyond the perimeter of the label gasket 1824 and the outer perimeter of the circuit board 1809 may additionally contact the lower surface of the label 1801.

The electronic components within the electronics assembly 1800 can utilize a flexible film layer 1802a that is bonded to the label 1801 forming a full enclosure for the electronic components and other parts. The electronic assembly 1800 can include a flexible film layer 1802a. The flexible film layer 1802a can be formed from materials as described herein with respect to the flexible film layers, for example, the flexible film layer 1802a can include a polymeric material, such as polyurethane, a paper material, or any other suitable material. In some cases, the flexible film layer 1802a can be a waterproof and/or gas tight film material. In some cases, the flexible film layer 1802a can be made of a polyurethane material (such as a thermoplastic polyurethane), polyester, ethylene-vinyl acetate, polyethylene, and/or any other suitable material.

In some cases, the flexible film layer 1802a and the lower surface of the label 1801 can be bonded or sealed around a perimeter. For example, the flexible film layer 1802a and the lower surface of the label 1801 can be bonded or sealed via heat and/or pressure around the perimeter or the two components can be melted or welded together forming a seal. In some cases, the flexible film layer 1802a and the lower surface of the label 1801 can be attached at a perimeter of the flexible film layer 1802a and lower surface of the label 1801 by heat welding, adhesive bonding, ultrasonic welding, RF welding, or any other attachment or bonding technique. FIG. 18 illustrates the perimeter 1842 of the flexible film layer 1802a as the line extending around the flexible film layer 1802a. FIG. 18 illustrates the perimeter 1844 of the label 1801 as the line extending around the perimeter of the label 1801. In some cases, the material of the label 1801 can make it difficult for the flexible film layer 1802a to be adhered to the label 1801. In some cases, the label 1801 or upper housing portion can be coated or treated with a material that allows for adhesion or sealing with the flexible film layer.

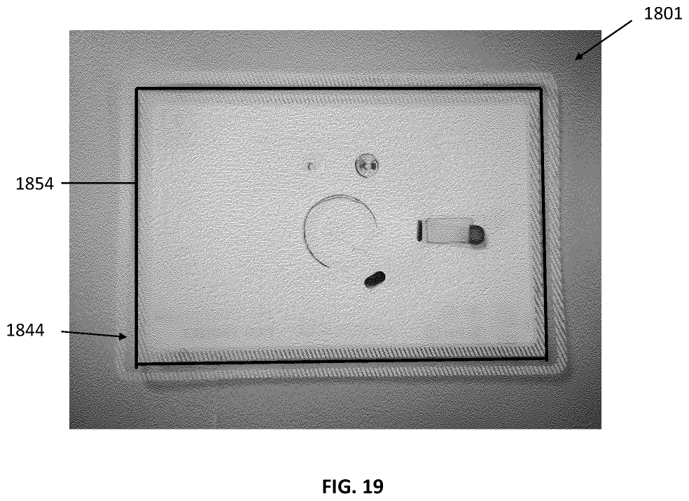

FIG. 19 illustrates an example of a label 1801 that can be used with the electronic assembly as described in FIG. 18. As shown in FIG. 19, a coating 1854 can be applied to a portion of the perimeter 1844 of the label 1801 on the lower side of the label 1801. The coating 1854 is shown in FIG. 19 as a line extending along the perimeter of the dressing. In some cases, the coating 1854 can be offset from the edge of the dressing along the perimeter as shown in FIG. 19. In some cases, the coating 1854 on the perimeter 1844 can be a polyurethane (PU) dispersion coating. In some cases, the coating 1854 on the perimeter 1844 can be a polyurethane (PU) dispersion coat or other dispersion coating that allows for adhesion or sealing of the material of the flexible film with the dispersion coating through the application of heat and/or pressure. The coating 1854 on a perimeter 1844 can allow the joining of the perimeter 1842 of the flexible film 1802a (shown in FIG. 18) and the perimeter 1844 of the label 1801 along the area of the coating 1854. In some cases, the perimeter 1842 of the flexible film 1802a can be attached to the perimeter 1844 of the label 1801 along the area of the coating 1854 by heat welding, adhesive bonding, ultrasonic welding, RF welding, or any other attachment or bonding technique.

In some cases, the coating along the perimeter of the label 1801 can replace the second flexible film 602b used in the electronics assembly 600 of FIG. 6. This can reduce the complexity of the electronics assembly as it reduces the layers and components that are assembled.

In some cases, the area of the coating can be limited to the border of the label and can be about 5 to 10 mm wide. In some cases, the width of coating area can be 2 mm to 14 mm (about 2 mm to 14 mm), 4 mm to 12 mm (about 4 mm to 12 mm), 5 mm to 10 mm (about 5 mm to 10 mm), or 6 mm to 8 mm (about 6 mm to 8 mm). The coating can be applied to the label 1801 material through coating application methods such as screen printing, spray coating, adhesive transfer, and/or other coating application techniques. The coating can allow for the joining or sealing of the flexible film layer to the label through the application of heat and pressure.

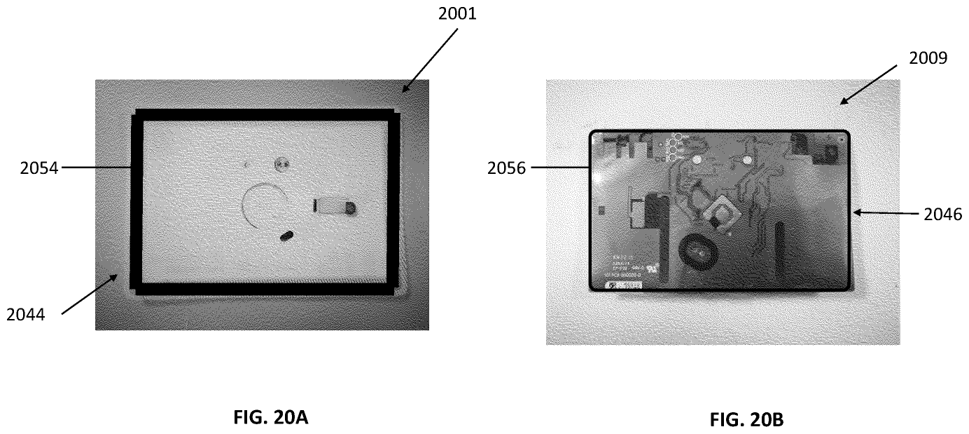

FIG. 20A-20B illustrates a configuration of a lower side of a label 2001 (FIG. 20A) and an upper side of a circuit board 2009 (FIG. 20B) with a coating around their perimeter. The label 2001 can include a coating 2054 on the perimeter 2044 of the label 2001. The coating 2054 is shown as the line around the perimeter of the label in FIG. 20A. The circuit board 2009 can include a coating 2056 on the perimeter 2046 of the circuit board 2009 as shown in FIG. 20B by the line around the perimeter of the circuit board 2009.

The coating 2054 and coating 2056 can be the same or similar to coating 1854 described with reference to FIGS. 18 and 19. The coating 2054 can coat the perimeter (shown with a black line around the perimeter in FIG. 20A) of the label 2001 joining with the upper surface of the perimeter area 1842 of the flexible film layer 1802a shown in FIG. 18 and the coating 2056 on the perimeter of the circuit board 2009 (shown as the black line following the edge of the circuit board) as shown in FIG. 20B. In some cases, the perimeter 1842 of the flexible film 1802a and the coating 2056 can be attached to the coating 2054 by heat welding, adhesive bonding, ultrasonic welding, RF welding, or any other attachment or bonding technique.

In some cases, the coatings 2054, 2056 along the perimeter of the label 2001 and the circuit board 2009 can replace the second flexible film 602b used in the electronics assembly 600 of FIG. 6 thereby reducing the complexity of the electronics assembly. As shown in FIG. 20A, the coating area of coating 2054 of the label 2001 can be limited to the border along the perimeter of the label 2001 and could be 15 to 20 mm wide. In some cases, the width of coating area can be 5 mm to 30 mm (about 5 mm to 30 mm), 10 mm to 25 mm (about 10 mm to 25 mm), or 15 to 20 mm (about 15 mm to 20 mm).

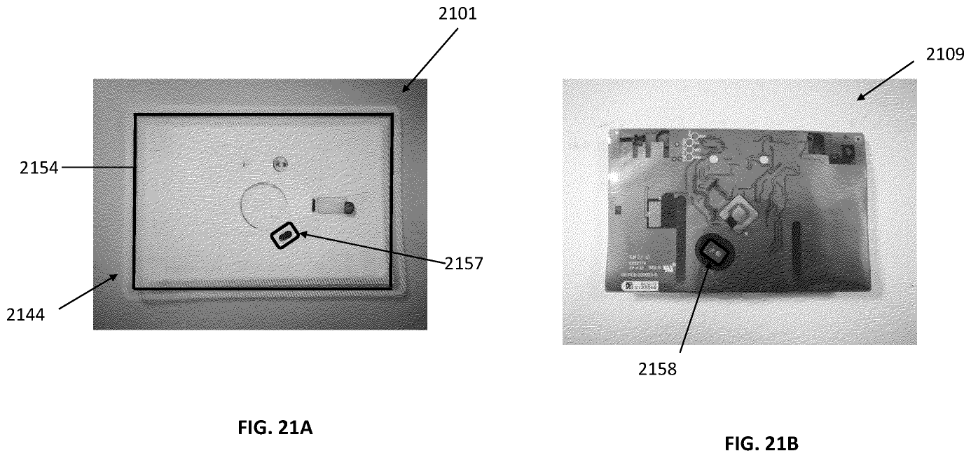

FIGS. 21A-21B illustrate a configuration of the lower side of a label 2101 (FIG. 21A) and an upper side of a circuit board 2109 (FIG. 21B). The label 2101 can include a first coating 2154 on the perimeter 2144 of the label 2101 and a second coating 2157 on the lower surface of the label around the exhaust hole of the label 2101. The first coating 2154 is shown as the line around the perimeter of the label and the second coating 2157 is shown as the line around the exhaust hole of the label in FIG. 21A. The circuit board 2109 can include a coating 2158 on the upper surface around the exhaust hole of the printed circuit board 2109 as shown in FIG. 21B by the line that extends around the exhaust hole of the circuit board 2109.

The coating 2154, 2157, and 2158 can be the same or similar to coating 1854 described with reference to FIGS. 18 and 19. The coating 2154 can coat the perimeter (shown as a black line around the label in FIG. 21A) of the label 2101 joining with the upper surface of the perimeter area 1842 of the flexible film layer 1802a shown in FIG. 18. In some cases, the coating 2154 can be attached to the upper surface of the perimeter area 1842 of the flexible film layer 1802a by heat welding, adhesive bonding, ultrasonic welding, RF welding, or any other attachment or bonding technique.

The coating 2158 on the on the upper surface around the exhaust hole of the printed circuit board 2109 (shown as the black line around the exhaust hole) as shown in FIG. 21B can be joined to the coating 2157 on the lower surface of the label 2101 around the exhaust hole of the label 2101 (shown as the black line around the exhaust hole) as shown in FIG. 21A. In some cases, the coating 2158 can be attached to the coating 2157 by heat welding, adhesive bonding, ultrasonic welding, RF welding, or any other attachment or bonding technique.

In some cases, the coating 2154 along the perimeter of the label 2101 and the coatings 2157, and 2158 around the exhaust holes of the label 2101 and the circuit board 2109 can replace the second flexible film 602b used in the electronics assembly 600 of FIG. 6 thereby reducing the complexity of the electronics assembly. As shown in FIG. 21A, the coating area of coating 2154 of the label 2101 can be limited to the border along the perimeter of the label 2101 and can be about 5 to 10 mm wide. In some cases, the width of coating area can be 2 mm to 14 mm (about 2 mm to 14 mm), 4 mm to 12 mm (about 4 mm to 12 mm), 5 mm to 10 mm (about 5 mm to 10 mm), or 6 mm to 8 mm (about 6 mm to 8 mm).

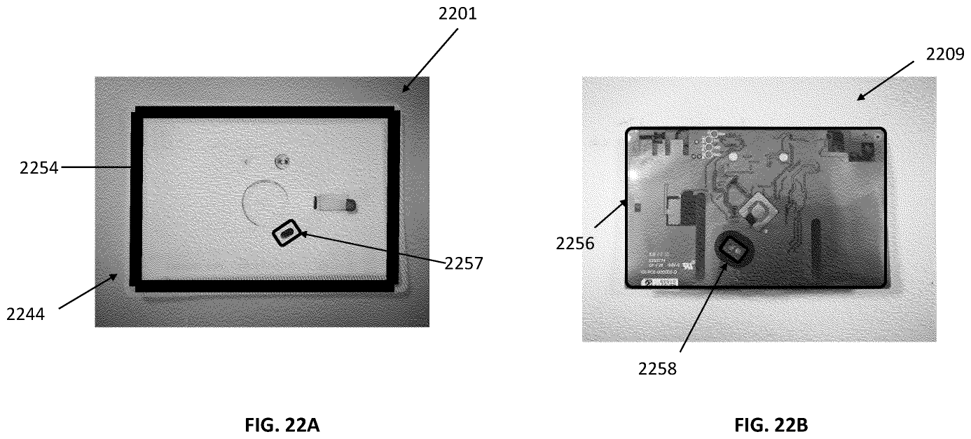

FIG. 22A-22B illustrates a configuration of the lower side of a label 2201 (FIG. 22A) and the upper side of a circuit board 2209 (FIG. 22B). The label 2101 can include a first coating 2254 on the perimeter 2244 of the label 2201 and a second coating 2257 on the label around the exhaust hole of the label 2201. The first coating 2254 is shown as the line around the perimeter of the label and the second coating 2257 is shown as the line around the exhaust hole of the label in FIG. 22A. The circuit board 2209 can include a first coating 2256 along the perimeter of the circuit board 2209 shown as the line around the circuit board in FIG. 22B and a second coating 2258 around the exhaust hole of the printed circuit board 2209 as shown in FIG. 22B by the line that extends around the exhaust hole of the circuit board 2209.

The coating 2254, 2257, 2256, and 2258 can be the same or similar to coating 1854 described with reference to FIGS. 18 and 19. The coating 2254 can coat the perimeter (shown as the black line around the perimeter of the label in FIG. 22A) of the label 2201 joining with the upper surface of the perimeter area 1842 of the flexible film layer 1802a shown in FIG. 18 and the coating 2256 on the perimeter of the circuit board 2209 (shown as the black line following the edge of the circuit board) as shown in FIG. 22B. In some cases, the coating 2254 can be attached to the upper surface of the perimeter area 1842 of the flexible film layer 1802a and the coating 2256 by heat welding, adhesive bonding, ultrasonic welding, RF welding, or any other attachment or bonding technique.

The coating 2258 on the on the upper surface around the exhaust hole of the printed circuit board 2209 (shown as the black line around the exhaust hole) as shown in FIG. 22B can be joined to the coating 2257 on the lower surface of the label 2201 around the exhaust hole of the label 2201 (shown as the black line around the exhaust hole) as shown in FIG. 22A. In some cases, the coating 2258 can be attached to the coating 2257 by heat welding, adhesive bonding, ultrasonic welding, RF welding, or any other attachment or bonding technique.

In some cases, the coatings 2254, 2256 along the perimeter of the label 2201 and the circuit board 2209 and the coatings 2257, 2258 around the exhaust holes of the label 2201 and the circuit board 2109 can replace the second flexible film 602b used in the electronics assembly 600 of FIG. 6 thereby reducing the complexity of the electronics assembly. As shown in FIG. 22A, the coating area of coating 2254 of the label 2201 can be limited to the border along the perimeter of the label 2001 and could be 15 to 20 mm wide. In some cases, the width of coating area can be 5 mm to 30 mm (about 5 mm to 30 mm), 10 mm to 25 mm (about 10 mm to 25 mm), or 15 to 20 mm (about 15 mm to 20 mm).

In some cases, the coatings along the perimeter of the circuit board and/or the coating along the perimeter of the exhaust holes of the circuit board can eliminate the need for a label gasket as the coatings can allow for sealing between the circuit board and the label around the perimeter and around the exhaust holes.

### Other Variations

While certain embodiments described herein relate to integrated negative pressure wound therapy systems in which the negative pressure source is supported by the dressing, systems and methods described herein are applicable to any negative pressure wound therapy system or medical system. For example, systems and methods for controlling operation described herein can be used in fluid-proof (such as, waterproof) negative pressure wound therapy systems or medical systems. Such systems can be configured with the negative pressure source and/or electronics being external to the wound dressing, such as with the negative pressure source and/or electronics being positioned in a fluid proof enclosure. Additionally, such systems can be configured to be used within ultrasound delivery devices, negative pressure devices powered by an external power supply, negative pressure devices with a separate pump, and medical devices generally.

Any of the embodiments disclosed herein can be used with one or more features disclosed in U.S. Pat. No. 7,779,625, titled “DEVICE AND METHOD FOR WOUND THERAPY,” issued Aug. 24, 2010; U.S. Pat. No. 7,964,766, titled “WOUND CLEANSING APPARATUS IN SITU,” issued on Jun. 21, 2011; U.S. Pat. No. 8,235,955, titled “WOUND TREATMENT APPARATUS AND METHOD,” issued on Aug. 7, 2012; U.S. Pat. No. 7,753,894, titled “WOUND CLEANSING APPARATUS WITH STRESS,” issued Jul. 13, 2010; U.S. Pat. No. 8,764,732, titled “WOUND DRESSING,” issued Jul. 1, 2014; U.S. Pat. No. 8,808,274, titled “WOUND DRESSING,” issued Aug. 19, 2014; U.S. Pat. No. 9,061,095, titled “WOUND DRESSING AND METHOD OF USE,” issued Jun. 23, 2015; U.S. Pat. No. 10,076,449, issued Sep. 18, 2018, titled “WOUND DRESSING AND METHOD OF TREATMENT”; U.S. patent application Ser. No. 14/418,908, filed Jan. 30, 2015, published as U.S. Publication No. 2015/0190286, published Jul. 9, 2015, titled “WOUND DRESSING AND METHOD OF TREATMENT”; U.S. Pat. No. 10,231,878, titled “TISSUE HEALING,” issued Mar. 19, 2019; PCT International Application PCT/GB2012/000587, titled “WOUND DRESSING AND METHOD OF TREATMENT” and filed on Jul. 12, 2012; International Application No. PCT/IB2013/001469, filed May 22, 2013, titled “APPARATUSES AND METHODS FOR NEGATIVE PRESSURE WOUND THERAPY”; PCT International Application No. PCT/IB2013/002102, filed Jul. 31, 2013, titled “WOUND DRESSING AND METHOD OF TREATMENT”; PCT International Application No. PCT/IB2013/002060, filed Jul. 31, 2013, titled “WOUND DRESSING AND METHOD OF TREATMENT”; PCT International Application No. PCT/IB2013/00084, filed Mar. 12, 2013, titled “REDUCED PRESSURE APPARATUS AND METHODS”; International Application No. PCT/EP2016/059329, filed Apr. 26, 2016, titled “REDUCED PRESSURE APPARATUSES”; PCT International Application No. PCT/EP2017/059883, filed Apr. 26, 2017, titled “WOUND DRESSINGS AND METHODS OF USE WITH INTEGRATED NEGATIVE PRESSURE SOURCE HAVING A FLUID INGRESS INHIBITION COMPONENT”; PCT International Application No. PCT/EP2017/055225, filed Mar. 6, 2017, titled “WOUND TREATMENT APPARATUSES AND METHODS WITH NEGATIVE PRESSURE SOURCE INTEGRATED INTO WOUND DRESSING”; PCT International Application No. PCT/EP2018/074694, filed Sep. 13, 2018, titled “NEGATIVE PRESSURE WOUND TREATMENT APPARATUSES AND METHODS WITH INTEGRATED ELECTRONICS”; PCT International Application No. PCT/EP2018/074701, filed Sep. 13, 2018, titled “NEGATIVE PRESSURE WOUND TREATMENT APPARATUSES AND METHODS WITH INTEGRATED ELECTRONICS”; PCT International Application No. PCT/EP2018/079345, filed Oct. 25, 2018, titled “NEGATIVE PRESSURE WOUND TREATMENT APPARATUSES AND METHODS WITH INTEGRATED ELECTRONICS”; PCT International Application No. PCT/EP2018/079745, filed Oct. 30, 2018, titled “SAFE OPERATION OF INTEGRATED NEGATIVE PRESSURE WOUND TREATMENT APPARATUSES”; each of which is incorporated by reference herein in its entirety.

Although certain embodiments described herein relate to wound dressings, systems and methods disclosed herein are not limited to wound dressings or medical applications. Systems and methods disclosed herein are generally applicable to electronic devices in general, such as electronic devices that can be worn by or applied to a user.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value. Moreover, although blocks of the various processes may be described in terms of determining whether a value meets or does not meet a particular threshold, the blocks can be similarly understood, for example, in terms of a value (i) being below or above a threshold or (ii) satisfying or not satisfying a threshold.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure.

The various components illustrated in the figures or described herein may be implemented as software or firmware on a processor, controller, ASIC, FPGA, or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A wound dressing apparatus comprising:
a cover layer configured to cover and form a seal over a wound; and
an electronics assembly comprising:
an electronics unit comprising a negative pressure source, wherein the negative pressure source comprises a body portion and an inlet extending from the body portion, wherein the inlet comprises a first end opposite a second end, wherein the second end is attached to the body portion; and
a housing comprising a plurality of flexible film layers, wherein the electronics unit is at least partially enclosed within the plurality of flexible film layers, wherein the plurality of flexible film layers comprises a first flexible film layer and a second flexible film layer, wherein the first and second flexible film layers are bonded together along a periphery of each of the first and second flexible film layers to at least partially enclose the electronics unit between the first and second flexible film layers, wherein the first flexible film layer comprises an aperture configured to receive the inlet and form a hermetic seal around the inlet to prevent wound exudate from entering into the electronics unit;
wherein the cover layer comprises an opening configured to receive the electronics assembly.

2. The wound dressing apparatus of claim 1, wherein the first end of the inlet comprises a first diameter and the second end of the inlet comprises a second diameter.

3. The wound dressing apparatus of claim 2, wherein the second diameter of the second end is greater than the first diameter of the first end so that the inlet tapers from the second end to the first end.

4. The wound dressing apparatus of claim 1, further comprising:
a wound contact layer comprising a proximal wound-facing face and a distal face, wherein the proximal wound-facing face is configured to be positioned in contact with the wound;
at least one absorbent layer over the wound contact layer;
wherein the cover layer is configured to cover and form a seal over the wound contact layer and the at least one absorbent layer; and
wherein the at least one absorbent layer comprises a recess configured to receive the electronics assembly.

5. The wound dressing apparatus of claim 1, wherein the electronics unit further comprises:
an outlet or exhaust mechanism positioned on an outlet of the negative pressure source, the outlet or exhaust mechanism comprising a vent aperture configured to expel air exhausted from the negative pressure source; and
a flexible circuit board, wherein the flexible circuit board comprises one or more of a sensor, a switch, a vent hole, and/or a light or LED indicators.

6. The wound dressing apparatus of claim 5, wherein the first flexible film layer comprises a second aperture configured to be aligned with the sensor on the circuit board, wherein the sensor is configured to measure the pressure from the inlet of the negative pressure source.

7. The wound dressing apparatus of claim 6, further comprising a filter configured to be positioned between the second aperture and the first sensor.

8. The wound dressing apparatus of claim 1, wherein the first flexible film layer and the second flexible film layer comprise a waterproof and/or gas tight film material.

9. The wound dressing apparatus of claim 1, wherein the first flexible film layer and the second flexible film layer comprise a polyurethane, a thermoplastic polyurethane, polyester, ethylene-vinyl acetate, or polyethylene.

10. The wound dressing apparatus of claim 1, wherein the first flexible film layer and the second flexible film layer comprises a material with a moisture vapor permeability configured to allow vapor to pass through the first and second flexible film layers.

11. The wound dressing apparatus of claim 1, wherein the housing further comprises an upper housing portion comprising a material layer forming a label for the electronics assembly, the upper housing portion comprises a first wound facing side and an opposite second side, wherein the second flexible film layer is bonded to the first side of the upper housing portion.

12. A wound dressing apparatus comprising:
a cover layer configured to cover and form a seal over a wound; and
an electronics assembly comprising:
an electronics unit comprising a negative pressure source; and
a housing comprising:
a lower flexible film layer comprising a first wound facing surface and an opposite second surface;
an upper housing portion comprising a first wound facing surface and an opposite second surface, the upper housing portion comprising a coating along a perimeter of the first surface of the upper housing portion;
wherein the electronics unit is at least partially enclosed within the flexible film layer and the upper housing portion; and
wherein the flexible film layer is bonded to the coating of the upper housing portion along a perimeter of the flexible film layer to at least partially enclose the electronics unit between the flexible film layer and the upper housing portion;
wherein the cover layer comprises an opening configured to receive the electronics assembly.

13. The wound dressing apparatus of claim 12, wherein the flexible film layer is bonded to the coating of the upper housing portion through heat and/or pressure.

14. The wound dressing apparatus of claim 12, further comprising a circuit board comprising an exhaust hole and a first wound facing surface and an opposite second surface.

15. The wound dressing apparatus of claim 14, wherein the coating of the upper housing portion comprises a first coating, wherein the circuit board comprises a second coating along a perimeter of the second surface of the circuit board, and wherein the first coating is bonded or sealed to the perimeter of the flexible film layer and the second coating on the circuit board.

16. The wound dressing apparatus of claim 14, wherein the coating of the upper housing portion comprises a first coating, the upper housing portion comprises a second coating on the first surface of the upper housing portion, wherein the second coating surrounds an exhaust hole in the upper housing portion, wherein the circuit board comprises a third coating on the second surface of the circuit board, the third coating surrounds the exhaust hole in the circuit board, and wherein the second coating of the upper housing portion is bonded to the third coating on the circuit board.

17. The wound dressing apparatus of claim 14, wherein the coating of the upper housing portion comprises a first coating, the upper housing portion comprises a second coating on the first surface of the upper housing portion, wherein the second coating surrounds an exhaust hole in the upper housing portion, wherein the circuit board comprises a third coating on the second surface of the circuit board, the third coating surrounds the exhaust hole in the circuit board, wherein the circuit board comprises a fourth coating along a perimeter of the second surface of the circuit board, and wherein the second coating of the upper housing portion is bonded to the third coating on the circuit board and the first coating is bonded to the perimeter of the flexible film layer and the fourth coating on the circuit board.

18. The wound dressing apparatus of claim 12, wherein the negative pressure source comprises a body portion and an inlet extending from the body portion, wherein the inlet comprises a first end opposite a second end, wherein the second end is attached to the body portion.

19. The wound dressing apparatus of claim 18, wherein the flexible film layer comprises an aperture configured to receive the inlet and form a hermetic seal around the inlet to prevent wound exudate from entering into the electronics unit.

20. The wound dressing apparatus of claim 12, wherein the coating comprises a dispersion coating, wherein the coating comprises a polyurethane (PU) dispersion coating.

21.-25. (canceled)
